(19) United States
(12) Patent Application Publication
(10) Pub. No.: US 2011/0293585 A1
(43) Pub. Date: Dec. 1, 2011

(54) COMPOSITIONS AND METHODS FOR TREATMENT OF LYSOSOMAL STORAGE DISORDERS

(75) Inventors: Jacob del Campo, North Haven, CT (US); Ranjit S. Bindra, New York, NY (US); Peter M. Glazer, Guilford, CT (US)

(73) Assignee: Helix Therapeutics, Inc.

(21) Appl. No.: 13/091,921

(22) Filed: Apr. 21, 2011

Related U.S. Application Data
(60) Provisional application No. 61/326,556, filed on Apr. 21, 2010.

(51) Int. Cl. A61K 48/00; A61P 3/00; C12Q 1/68; A61P 43/00; C07H 21/00; A61K 38/08

(52) U.S. Cl. 424/93.21; 514/44 R; 536/23.1; 514/21.8; 435/6.18

(57) ABSTRACT

Compositions and methods for treating lysosomal storage diseases are disclosed. Lysosomal dysfunction is usually the result of deficiency of a single enzyme necessary for the metabolism of lipids, glycoproteins (sugar containing proteins) or mucopolysaccharides which are fated for breakdown or recycling. The compositions contain triplex-forming molecules which can be used to induce site-specific homologous recombination in mammalian cells when combined with donor DNA molecules, by stimulating cellular DNA synthesis, recombination, and repair mechanisms. The methods are particular useful for correcting point mutations in genes associated with lysosomal storage diseases such as Gaucher's disease, Fabry disease, and Hurler syndrome. Methods for determining the frequency of target gene repair and assessing the restoration of the enzymatic activity of corrected polypeptides are also disclosed. Ex vivo and in vivo methods of gene correction in patients are also provided.

### CROSS-REFERENCE TO RELATED APPLICATIONS

This application claims benefit of and priority to U.S. Ser. No. 61/326,556, filed Apr. 21, 2010, which is incorporated by reference in its entirety.

### FIELD OF THE INVENTION

The present disclosure generally relates to the field of compositions and methods for targeted correction of mutations in genes encoding enzymes necessary for the metabolism of lipids, glycoproteins, or mucopolysaccharides.

### REFERENCE TO SEQUENCE LISTING

The Sequence Listing being submitted herewith as a text file named “HT_{—}100_ST25.txt,” created on Apr. 20, 2011, and having a size of 7,079 bytes is hereby incorporated by reference pursuant to 37 C.F.R. §1.52(e)(5).

### BACKGROUND OF THE INVENTION

Lysosomal storage diseases (LSDs) are a group of more than 50 clinically-recognized, rare inherited metabolic disorders that result from defects in lysosomal function (Walkley, J. Inherit. Metab. Dis., 32(2):181-9 (2009)). Lysosomal storage disorders are caused by dysfunction of the cell's lysosome orangelle, which is part of the larger endosomal/lysosomal system. Together with the ubiquitin-proteosomal and autophagosomal systems, the lysosome is essential to substrate degradation and recycling, homeostatic control, and signaling within the cell. Lysosomal dysfunction is usually the result of a deficiency of a single enzyme necessary for the metabolism of lipids, glycoproteins (sugar containing proteins) or mucopolysaccharides (long unbranched polysaccharides consisting of a repeating disaccharide unit; also known as glycosaminoglycans, or GAGs) which are fated for breakdown or recycling. Enzyme deficiency reduces or prevents break down or recycling of the unwanted lipids, glycoproteins, and GAGs, and results in buildup or “storage” of these materials within the cell. Most lysosomal diseases show widespread tissue and organ involvement, with brain, viscera, bone and connective tissues often being affected. More than two-thirds of lysosomal diseases affect the brain. Neurons appear particularly vulnerable to lysosomal dysfunction, exhibiting a range of defects from specific axonal and dendritic abnormalities to neuron death.

Individually, LSDs occur with incidences of less than 1:100,000, however, as a group the incidence is as high as 1 in 1,500 to 7,000 live births (Staretz-Chacham, et al., Pediatrics, 123(4):1191-207 (2009)). LSDs are typically the result of inborn genetic errors. Most of these disorders are autosomal recessively inherited, however a few are X-linked recessively inherited, such as Fabry disease and Hunter syndrome (MPS II). Affected individuals generally appear normal at birth, however the diseases are progressive. Develop of clinical disease may not occur until years or decades later, but is typically fatal. Lysosomal storage diseases affect mostly children and they often die at a young and unpredictable age, many within a few months or years of birth. Many other children die of this disease following years of suffering from various symptoms of their particular disorder. Clinical disease may be manifest as mental retardation and/or dementia, sensory loss including blindness or deafness, motor system dysfunction, seizures, sleep and behavioral disturbances, and so forth. Some people with Lysosomal storage disease have enlarged livers (hepatomegaly) and enlarged spleens (splenomegaly), pulmonary and cardiac problems, and bones that grow abnormally.

Treatment for many LSDs is enzyme replacement therapy (ERT) and/or substrate reduction therapy (SRT), as wells as treatment or management of symptoms. The average annual cost of ERT in the United States ranges from $90,000 to $565,000. While ERT has significant systemic clinical efficacy for a variety of LSDs, little or no effects are seen on central nervous system (CNS) disease symptoms, because the recombinant proteins cannot penetrate the blood-brain barrier. Allogeneic hematopoietic stem cell transplantation (HSCT) represents a highly effective treatment for selected LSDs. It is currently the only means to prevent the progression of associated neurologic sequelae. However, HSCT is expensive, requires an HLA-matched donor and is associated with significant morbidity and mortality. Recent gene therapy studies suggest that LSDs are good targets for this type of treatment.

Gene therapy can be defined by the methods used to introduce heterologous DNA into a host cell or by the methods used to alter the expression of endogenous genes within a cell. As such, gene therapy methods can be used to alter the phenotype and/or genotype of a cell.

Targeted modification of the genome by gene replacement is of value as a research tool and in gene therapy. However, while facile methods exist to introduce new genes into mammalian cells, the frequency of homologous integration is limited (Hanson et al., (1995) Mol. Cell. Biol. 15(1), 45-51), and isolation of cells with site-specific gene insertion typically requires a selection procedure (Capecchi, M. R., (1989) Science 244(4910), 1288-1292). Site-specific DNA damage in the form of double-strand breaks produced by rare cutting endonucleases can promote homologous recombination at chromosomal loci in several cell systems, but this approach requires the prior insertion of the recognition sequence into the locus.

Methods which alter the genotype of a cell typically rely on the introduction into the cell of an entire replacement copy of a defective gene, a heterologous gene, or a small nucleic acid molecule such as an oligonucleotide, to treat human, animal and plant genetic disorders. The introduced gene or nucleic acid molecule, via genetic recombination, replaces the endogenous gene. This approach requires complex delivery systems to introduce the replacement gene into the cell, such as genetically engineered viruses, or viral vectors.

Alternatively, gene therapy methods can be used to alter the expression of an endogenous gene. One example of this type of method is antisense therapy. In antisense therapy, a nucleic acid molecule is introduced into a cell, the nucleic acid molecule being of a specific nucleic acid sequence so as to hybridize or bind to the mRNA encoding a specific protein. The binding of the antisense molecule to an mRNA species decreases the efficiency and rate of translation of the mRNA.

Gene therapy is being used on an experimental basis to treat well known genetic disorders of humans such as retinoblastoma, cystic fibrosis, and globinopathies such as sickle cell anemia. However, in vivo efficiency is low due to the limited number of recombination events actually resulting in replacement of the defective gene.

Gene therapy approaches have yielded the most promising pre-clinical efficacy data for the treatment of the lysosomal storage disease Hurler Syndrome (HS). However, the therapies use viral vectors to introduce expression constructs rather than by correcting the intrinsic gene itself This approach has significant weaknesses. Viral vector integration occurs in a non-targeted manner, often resulting in an inadequate therapeutic effect and/or toxicity. In terms of toxicity, insertion at inappropriate sites causes problems, as occurred in a gene therapy trial in which two patients developed leukemia, apparently due to retroviral vector integration at the LMO-2 locus (Hacein-Bey-Abina et. al, Science 301:5644 (2003)). Autologous HSCT requires a large number of CD34+ cells, typically in the range of 50-100 million cells. Thus, even if the frequency of insertional mutagenesis is low (e.g., 0.01%), one can expect up to 10,000 potentially deleterious mutations in a single gene therapy treatment.

Alternative approaches include the use of artificial nucleases with engineered binding domains including zinc finger DNA-binding domains fused to the nuclease domain of the FokI restriction enzyme (ZFNs). ZFNs are attractive because they can induce site-specific gene modification at high frequencies. However, off-target cleavage remains an issue. ZFNs are advantageous because they can induce site-specific gene modification at high frequencies. However, due to the size of artificial nucleases, they must be delivered by viral or plasmid vectors, thus re-introducing concerns of the delivery technology. The minimization of off-target cleavage events is essential when working with undifferentiated stem cells. These cells have a substantial proliferative capacity, and random mutations in these cells can have a substantial risk of inducing leukemogenesis. Similar to the case of gene therapy and insertional mutagenesis, even a low rate of off-target cleavage events can have serious risks with regard to oncogenic transformation, given the large numbers stem cells that would be subjected to ZFN-induced DNA damage and repair.

Short-fragment homologous recombination (SFHR) and the use of 40- to 60-mer DNA olignucleotides are additional non-viral approaches to gene correction which have been proposed. However, a limitation of these approaches is the lack of a method to stimulate the recombination event. For example, SFHR is likely to be more specific than ZFNs with regard to gene targeting. However, this approach is not practical for developing a therapy because of the low efficiency of the technique (ranging between 0.1 and 1%) without a means to stimulate recombination, and there is currently no strategy to increase the efficacy.

Since the initial observation of triple-stranded DNA many years ago by Felsenfeld et al., J. Am. Chem. Soc. 79:2023 (1957), oligonucleotide-directed triple helix formation has emerged as a valuable tool in molecular biology. Current knowledge suggests that oligonucleotides can bind as third strands of DNA in a sequence specific manner in the major groove in polypurine/polypyrimidine stretches in duplex DNA. In one motif, a polypyrimidine oligonucleotide binds in a direction parallel to the purine strand in the duplex, as described by Moser and Dervan, Science 238:645 (1987), Praseuth et al., Proc. Natl. Acad. Sci. USA 85:1349 (1988), and Mergny et al., Biochemistry 30:9791 (1991). In the alternate purine motif, a polypurine strand binds anti-parallel to the purine strand, as described by Beal and Dervan, Science 251:1360 (1991). The specificity of triplex formation arises from base triplets (AAT and GGC in the purine motif) formed by hydrogen bonding; mismatches destabilize the triple helix, as described by Mergny et al., Biochemistry 30:9791 (1991) and Beal and Dervan, Nuc. Acids Res. 11:2773 (1992).

Triplex forming oligonucleotides (TFOs) are useful for several molecular biology techniques. For example, triplex forming oligonucleotides designed to bind to sites in gene promoters have been used to block DNA binding proteins and to block transcription both in vitro and in vivo. (Maher et al., Science 245:725 (1989), Orson et al., Nucleic Acids Res. 19:3435 (1991), Postal et al., Proc. Natl. Acad. Sci. USA 88:8227 (1991), Cooney et al., Science 241:456 (1988), Young et al., Proc. Natl. Acad. Sci. USA 88:10023 (1991), Maher et al., Biochemistry 31:70 (1992), Duval-Valentin et al., Proc. Natl. Acad. Sci. USA 89:504 (1992), Blume et al., Nucleic Acids Res. 20:1777 (1992), Durland et al., Biochemistry 30:9246 (1991), Grigoriev et al., J. of Biological Chem. 267:3389 (1992), and Takasugi et al., Proc. Natl. Acad. Sci. USA 88:5602 (1991)). Site specific cleavage of DNA has been achieved by using triplex forming oligonucleotides linked to reactive moieties such as EDTA-Fe(II) or by using triplex forming oligonucleotides in conjunction with DNA modifying enzymes (Perrouault et al., Nature 344:358 (1990), Francois et al., Proc. Natl. Acad. Sci. USA 86:9702 (1989), Lin et al., Biochemistry 28:1054 (1989), Pei et al., Proc. Natl. Acad. Sci. USA 87:9858 (1990), Strobel et al., Science 254:1639 (1991), and Posvic and Dervan, J. Am. Chem Soc. 112:9428 (1992)). Sequence specific DNA purification using triplex affinity capture has also been demonstrated. (Ito et al., Proc. Natl. Acad. Sci. USA 89:495 (1992)). Triplex forming oligonucleotides linked to intercalating agents such as acridine, or to cross-linking agents, such as p-azidophenacyl and psoralen, have been utilized. (Praseuth et al., Proc. Natl. Acad. Sci. USA 85:1349 (1988), Grigoriev et al., J. of Biological Chem. 267:3389 (1992), Takasugi et al., Proc. Natl. Acad. Sci. USA 88:5602 (1991).

Methods for targeted gene therapy using triplex-forming oligonucleotides (TFOs) and peptide nucleic acids (PNAs) are described in U.S. Application No. 20070219122 and their use for treating infectious diseases such as HIV are described in U.S. Application No. 2008050920, however there remains a need to identify viable compositions and methods for gene therapy mediated modification of genes associated with lysosomal storage disease such as Gaucher's disease, Hurler's disease, and Fabry's disease.

It is therefore an object of the invention to provide safe, non-toxic compositions and methods for targeted gene correction of correction of mutations in genes encoding enzymes necessary for the metabolism of lipids, glycoproteins, or mucopolysaccharides.

It is a further object of the invention to provide compositions and methods for targeted gene correction of the W402X and Q70X mutations in the human α-L-iduronidase gene, and restore enzyme function.

It is another object of the invention to provide methods for identifying successful correction of the target gene by measuring enzyme activity in isolated protein.

It is a further object of the invention to provide methods for determining the frequency of induced recombination in a treated population of cells by measuring the enzyme activity in isolated protein.

### SUMMARY OF THE INVENTION

Lysosomal storage disorders are caused by dysfunction of the cell's lysosome orangelle, which is part of the larger endosomal/lysosomal system. Lysosomal dysfunction is usually the result of deficiency of a single enzyme necessary for the metabolism of lipids, glycoproteins (sugar containing proteins) or mucopolysaccharides which are fated for breakdown or recycling. Enzyme deficiency reduces or prevents break down or recycling of the unwanted lipids, glycoproteins, and GAGs, and results in buildup or “storage” of these materials within the cell.

Compositions and methods for treating lysosomal storage diseases have been developed. The compositions contain “triplex-forming molecules,” that bind to duplex DNA in a sequence-specific manner to form a triple-stranded structure. Triplex-forming molecules include, but are not limited to, triplex-forming oligonucleotides (TFOs), peptide nucleic acids (PNA), and “tail clamp” PNA (tcPNA). The triplex-forming molecules can be used to induce site-specific homologous recombination in mammalian cells when combined with donor DNA molecules, by stimulating cellular DNA synthesis, recombination, and repair mechanisms.

Methods for introducing mutations into the target duplex DNA using triplex-forming molecules and donor oligonucleotides are also disclosed. The methods are particular useful for correcting mutations in genes associated with lysosomal storage diseases such as Gaucher's disease, Fabry disease, and Hurler syndrome. If the target gene contains a mutation, such as a non-sense point mutation that results in dysfunction of an enzyme encoded by the target gene, then compositions containing triplex-forming molecules and donor oligonucleotides are useful for mutagenic repair that restores the wildtype DNA sequence of the target gene. Repair of the endogenous gene partially or completely restores the function of the encoded enzyme.

Examples demonstrate tail clamp peptide nucleic acids and donor oligonucleotides designed to correct the W402X and Q70X mutations in the human α-L-iduronidase gene, and restore enzyme function have been developed. Methods for determining the frequency of target gene repair and assessing the restoration of the enzymatic activity of corrected polypeptides are also disclosed.

Ex vivo and in vivo methods of gene correction in patients are also disclosed. For ex vivo gene therapy, cells are isolated from a subject and contacted ex vivo with the compositions to produce cells containing mutations in or adjacent to genes. The corrected cells are then returned to the patient to reduce, alleviate, or cure the disorder. The disclosed compositions including triplex-forming molecules can also be employed for therapeutic uses in vivo in combination with a suitable pharmaceutical carrier.

### BRIEF DESCRIPTION OF THE DRAWINGS

FIG. 1 is a flowchart showing the experimental design employed to modify the α-L-iduronidase gene (IDUA) gene in cells.

FIG. 2 is a schematic showing the binding of tail clamp peptide nucleic acid (PNA-70, top) having the generic sequence JJT TJT-EEE-TCT TCC GAG CAG (SEQ ID NO: 29) designed to bind to a polypurine target site with the sequence 5′ CTGCTCGGAAGA 3′(SEQ ID NO: 2) which is a subsequence of 5′ TGGGGGCTGCTCGGAAGACCCCTT 3′ (SEQ ID NO: 3) located 170 base pairs downstream of the Q70X mutation. The compliment of SED ID NO: 3 is 5′AAGGGGTCTTCCGAGCAGCCCCCA (SEQ ID NO: 4). Also shown is binding of tail clamp peptide nucleic acid (PNA-402, bottom) having the generic sequence T TJJ JJT-EEE-TCC CCT TGG TGA AGG (SEQ ID NO: 30) designed to bind to a polypurine target site with the sequence 5′ CCTTCACCAAGGGGA 3′ (SEQ ID NO: 6) which is a subsequence of 5′ GGGACTCCTTCACCAAGGGGAGGGGGA 3′ (SEQ ID NO:7) located 100 base pairs upstream of the W402X mutation. The compliment of SEQ ID NO: 7 is 5′ TCCCCCTCCCCTTGGTGAAGGAGTCCC 3′ (SEQ ID NO: 8). J=pseudoisocytosine and E=flexible linker PNA sequences are from N-terminus to C-terminus.

FIG. 3 is a diagram of the α-L-iduronidase gene (IDUA) gene, including the common Q70X and W402X and their relative locations within exon 2 and exon 9 respectively, and the location of two triplex binding sites, one within exon 2 (approximately 170 base pairs downstream of the Q70X mutation) and another upstream of exon 9 (approximately 100 base pairs upstream of the W402X mutation).

FIG. 4 is a schematic of the allele-specific PCR strategy to detect modifications in the endogenous IDUA gene. Relative positions of the gene-specific forward “F” primer, the allele-specific forward “F” primer, the gene-specific reverse “R” primer, and the antisense donor oligonucleotide are labeled. Point mutations in the allele-specific forward primer and the antisense donor oligonucleotide are depicted with short vertical lines, and the location of the non-sense mutation in the mutant gene is indicated with a single longer vertical line. The sequence of a stretch of the IDUA gene containing the Q70X mutation (5′ CTCAGCTGGGACTAGCAGCTCAACCTC 3′ (SEQ ID NO: 9)) and the sequence changes introduced by a wildtype codon modifier (WT CM) (5′ TTAAGCTGGGATCAGCAATTGAATTTG 3′ SEQ ID NO: 10)) are compared as an example. Sequence changes introduced by the wildtype codon modifier relative to the Q70X sequence are underlined.

FIG. 5 shows the sequence of a stretch of the IDUA gene containing the W402X mutation (5′ GAGGAGCAGCTCTAGGCCGAA 3′ (SEQ ID NO: 11)) and the sequence changes introduced by a wildtype codon modifier (WT CM) (5′ GAAGAACAATTATGGGCGGAA 3′ (SEQ ID NO: 12)). Sequence changes introduced by the wildtype codon modifier relative to the W402X sequence are underlined.

FIG. 6 is a line graph (standard curve) showing the pg IDUA activity/μg protein for protein samples from cell populations of increasing ratio of heterozygous W402X+/−human primary fibroblasts mixed with homozygous W402−/−fibroblasts (2:98, 5:95, 10:90, 25:75, and 50:50), giving final WT allele frequencies of 1%, 2.5%, 5%, 12.5%, and 25%, respectively, as labeled.

FIG. 7 is a bar graph showing the IDUA enzyme activity ((pg/μg total protein) relative to 4MU standard curve amounts) for 1%, 2.5%, 5%, 10%, 12.5%, and 25% wildtype protein (standard curve); and protein from IDUA homozygous mutant, IDUA heterozygous, and IDUA homozygous wildtype samples.

FIG. 8 is a bar graph showing the relative IDUA activity (pg/hr/μg protein) in protein samples isolated W402−/−fibroblasts either mock transfected, transfected with 4 μM W402XCM donor oligonucleotide/4 μM PNA-402tc715, or transfected with 6 μM W402XCM donor oligonucleotide/4 μM PNA-402tc715.

FIG. 9 is a bar graph showing the relative allele frequency, as determined by IDUA activity, of wildtype cells, W402−/−fibroblasts, W402−/−fibroblasts transfected with 4 μM W402XCM donor oligonucleotide/4 μM PNA-402tc715, and W402−/−fibroblasts transfected with 6 μM W402XCM donor oligonucleotide/4 μM PNA-402tc715.

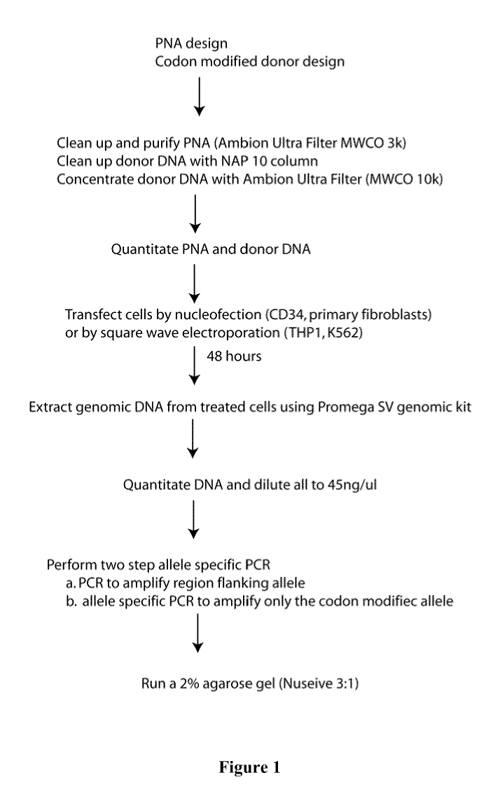

### DETAILED DESCRIPTION OF THE INVENTION

I. Compositions that bind to Double-Stranded DNA Encoding Lysosomal Storage Disease Gene

Compositions containing “triplex-forming molecules,” that bind to duplex DNA in a sequence-specific manner to form a triple-stranded structure include, but are not limited to, triplex-forming oligonucleotides (TFOs), peptide nucleic acids (PNA), and “tail clamp” PNA (tcPNA). The triplex-forming molecules can be used to induce site-specific homologous recombination in mammalian cells when combined with donor DNA molecules. The donor DNA molecules can contain mutated nucleic acids relative to the target DNA sequence. This is useful to activate, inactivate, or otherwise alter the function of a polypeptide or protein encoded by the targeted duplex DNA. Triplex-forming molecules include triplex-forming oligonucleotides and peptide nucleic acids.

A. Triplex-Forming Molecules

1. Triplex-Forming Oligonucleotides (TFOs)

Triplex-forming oligonucleotides (TFOs) are defined as oligonucleotides which bind as third strands to duplex DNA in a sequence specific manner. The oligonucleotides are synthetic or isolated nucleic acid molecules which selectively bind to or hybridize with a predetermined target sequence, target region, or target site within or adjacent to a human gene such as encoding the α-L-iduronidase gene so as to form a triple-stranded structure.

Preferably, the oligonucleotide is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful.

The oligonucleotides are preferably generated using known DNA synthesis procedures. In one embodiment, oligonucleotides are generated synthetically. Oligonucleotides can also be chemically modified using standard methods that are well known in the art.

The nucleotide sequence of the oligonucleotides is selected based on the sequence of the target sequence, the physical constraints imposed by the need to achieve binding of the oligonucleotide within the major groove of the target region, and the need to have a low dissociation constant (K_{d}) for the oligonucleotide/target sequence. The oligonucleotides have a base composition which is conducive to triple-helix formation and is generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C⁺.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

Preferably, the oligonucleotide binds to or hybridizes to the target sequence under conditions of high stringency and specificity. Most preferably, the oligonucleotides bind in a sequence-specific manner within the major groove of duplex DNA. Reaction conditions for in vitro triple helix formation of an oligonucleotide probe or primer to a nucleic acid sequence vary from oligonucleotide to oligonucleotide, depending on factors such as oligonucleotide length, the number of G:C and A:T base pairs, and the composition of the buffer utilized in the hybridization reaction. An oligonucleotide substantially complementary, based on the third strand binding code, to the target region of the double-stranded nucleic acid molecule is preferred.

As used herein, an oligonucleotide is said to be substantially complementary to a target region when the oligonucleotide has a heterocyclic base composition which allows for the formation of a triple-helix with the target region. As such, an oligonucleotide is substantially complementary to a target region even when there are non-complementary bases present in the oligonucleotide. As stated above, there are a variety of structural motifs available which can be used to determine the nucleotide sequence of a substantially complementary oligonucleotide.

2. Peptide Nucleic Acids (PNA)

In another embodiment, the triplex-forming molecules are peptide nucleic acids (PNAs). Peptide nucleic acids are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that are similar to oligonucleotides, but are achiral and neutrally charged molecules. Peptide nucleic acids are comprised of peptide nucleic acid monomers. The heterocyclic bases can be any of the standard bases (uracil, thymine, cytosine, adenine and guanine) or any of the modified heterocyclic bases described below.

PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules, or two PNA molecules linked together by a linker of sufficient flexibility to form a single bis-PNA molecule. In both cases, the PNA molecule(s) forms a triplex “clamp” with one of the strands of the target duplex while displacing the other strand of the duplex target. In this structure, one strand forms Watson-Crick base pairs with the DNA strand in the anti-parallel orientation (the Watson-Crick binding portion), whereas the other strand forms Hoogsteen base pairs to the DNA strand in the parallel orientation (the Hoogsteen binding portion). A homopurine strand allows formation of a stable PNA/DNA/PNA triplex. PNA clamps can form at shorter homopurine sequences than those required by triplex-forming oligonucleotides (TFOs) and also do so with greater stability.

Suitable molecules for use in linkers of bis-PNA molecules include, but are not limited to, 8-amino-3,6-dioxaoctanoic acid, referred to as an O-linker, and 6-aminohexanoic acid. Poly(ethylene)glycol monomers can also be used in bis-PNA linkers. A bis-PNA linker can contain multiple linker molecule monomers in any combination.

PNAs can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy or the N-terminus of a PNA strand.

3. Tail Clamp Peptide Nucleic Acids (tcPNA)

Although polypurine:polypyrimidine stretches do exist in mammalian genomes, it is desirable to target triplex formation in the absence of this requirement. In some embodiments such as PNA, triplex-forming molecules include a “tail” added to the end of the Watson-Crick binding portion. Adding additional nucleobases, known as a “tail” or “tail clamp”, to the Watson-Crick binding portion that bind to the target strand outside the triple helix further reduces the requirement for a polypurine:polypyrimidine stretch and increases the number of potential target sites. The tail is most typically added to the end of the Watson-Crick binding sequence furthest from the linker. This molecule therefore mediates a mode of binding to DNA that encompasses both triplex and duplex formation (Kaihatsu, et al., Biochemistry, 42(47):13996-4003 (2003); Bentin, et al., Biochemistry, 42(47):13987-95 (2003)). For example, if the triplex-forming molecules are tail clamp PNA (tcPNA), the PNA/DNA/PNA triple helix portion and the PNA/DNA duplex portion both produce displacement of the pyrimidine-rich strand, creating an altered helical structure that strongly provokes the nucleotide excision repair pathway and activating the site for recombination with a donor DNA molecule (Rogers, et al., Proc. Natl. Acad. Sci. U.S.A., 99(26):16695-700 (2002)). Tail clamps added to PNAs (referred to as tcPNAs) have been described by Kaihatsu, et al., Biochemistry, 42(47):13996-4003 (2003); Bentin, et al., Biochemistry, 42(47):13987-95 (2003), and are known to bind to DNA more efficiently due to low dissociation constants. The addition of the tail also increases binding specificity and binding stringency of the triplex-forming molecules to the target duplex. It has also been found that the addition of a tail to clamp PNA improves the frequency of recombination of the donor oligonucleotide at the target site compared PNA without the tail.

4. Chemical Modifications

The triplex-forming molecules including TFOs, PNAs and other suitable oligonucleotides, may include one or more modifications or substitutions to the nucleobases, sugars, or linkages. Under physiologic conditions, potassium levels are high, magnesium levels are low, and pH is neutral. These conditions are generally unfavorable to allow for effective binding of TFOs to duplex DNA. For example, high potassium promotes guanine (G)-quartet formation, which inhibits the activity of G-rich purine motif TFOs. Also, magnesium, which is present at low concentrations under physiologic conditions, supports third-strand binding by charge neutralization. Finally, neutral pH disfavors cytosine protonation, which is needed for pyrimidine motif third-strand binding. Target sequences with adjacent cytosines are particularly problematic. Triplex stability is greatly compromised by runs of cytosines, thought to be due to repulsion between the positive charge resulting from the N³ protonation or perhaps because of competition for protons by the adjacent cytosines.

Chemical modification of nucleotides comprising triplex-forming molecules may be useful to increase binding affinity of triplex-forming molecules and/or triplex stability under physiologic conditions. Modified nucleotides may comprise one or more of the nucleotides which comprise a triplex-forming oligonucleotide or peptide nucleic acid. As used herein “modified nucleotide” or “chemically modified nucleotide” defines a nucleotide that has a chemical modification of one or more of the heterocyclic base, sugar moiety or phosphate moiety constituents. Preferably the charge of the modified nucleotide is reduced compared to DNA or RNA oligonucleotides of the same nucleobase sequence. Most preferably the triplex-forming molecules have low negative charge, no charge, or positive charge such that electrostatic repulsion with the nucleotide duplex at the target site is reduced compared to DNA or RNA oligonucleotides with the corresponding nucleobase sequence. Preferably, modified oligonucleotides in TFOs are able to form Hoogsteen and/or reverse Hoogsteen base pairs with bases of the target sequence. More preferably, modified oligonucleotides increase the binding affinity of the TFO to the target duplex DNA, or the stability of the formed triplex. Modifications should not prevent, and preferably enhance and/or stabilize, triplex formation as described above by increasing specificity or binding affinity of the triplex-forming molecules to the target site. Modified bases and base analogues, modified sugars and sugar analogues and/or various suitable linkages known in the art are also suitable for use in triplex-forming molecules.

Examples of modified nucleotides with reduced charge include modified internucleotide linkages such as phosphate analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

a. Heterocyclic Bases

The principal naturally-occurring nucleotides comprise uracil, thymine, cytosine, adenine and guanine as the heterocyclic bases. Chemical modifications of heterocyclic bases or heterocyclic base analogs may be effective to increase the binding affinity of a nucleotide or its stability in a triplex. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine(2-aminopyridine), and various pyrrolo- and pyrazolopyrimidine derivatives. Substitution of 5-methylcytosine or pseudoisocytosine for cytosine in triplex-forming molecules helps to stabilize triplex formation at neutral and/or physiological pH, especially in triplex-forming molecules with isolated cytosines. This is because the positive charge partially reduces the negative charge repulsion between the triplex-forming molecules and the target duplex, and allows for Hoogsteen binding. Substitutions of 2′-O-methylpseudocytidine for cytidine are especially useful to stabilize triplexes formed by TFOs and target duplexes when the target sequence contains adjacent cytidines.

b. Sugars

Triplex-forming oligonucleotides may also contain nucleotides with modified sugar moieties or sugar moiety analogs. Sugar moiety modifications include, but are not limited to, 2′-O-aminoethoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O-(N-(methyl)acetamido) (2′-OMA). 2′-O-aminoethyl sugar moiety substitutions are especially preferred because they are protonated at neutral pH and thus suppress the charge repulsion between the TFO and the target duplex. This modification stabilizes the C3′-endo conformation of the ribose or dexyribose and also forms a bridge with the i-1 phosphate in the purine strand of the duplex.

c. Internucleotide Linkages

The nucleotide subunits of the triplex-forming molecules are connected by an internucleotide bond that refers to a chemical linkage between two nucleoside moieties. Modifications to the phosphate backbone of triplex-forming oligonucleotides may also increase the binding affinity of TFOs or stabilize the triplex formed between the TFO and the target duplex. Cationic modifications, including, but not limited to, diethyl-ethylenediamide (DEED) or dimethyl-aminopropylamine (DMAP) may be especially useful due to decrease electrostatic repulsion between TFO and duplex target phosphates. Modifications of the phosphate backbone may also include the substitution of a sulfur atom for one of the non-bridging oxygens in the phosphodiester linkage. This substitution creates a phosphorothioate internucleoside linkage in place of the phosphodiester linkage. Oligonucleotides containing phosphorothioate internucleoside linkages have been shown to be more stable in vivo.

Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds, which allow them to form PNA-DNA or PNA-RNA duplexes via Watson-Crick base pairing with high affinity and sequence-specificity. PNAs maintain spacing of heterocyclic bases that is similar to conventional DNA oligonucleotides, but are achiral and neutrally charged molecules. Peptide nucleic acids are comprised of peptide nucleic acid monomers.

Other backbone modifications or constituents of triplex-forming molecules, particularly those relating to PNAs, include peptide and amino acid variations and modifications. Thus, the backbone of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Triplex-forming molecules such as PNAs may optionally include one or more terminal amino acids at either or both termini to increase stability, and/or affinity of the PNAs or modified nucleotides for DNA, or increase solubility of PNAs or modified nucleotides. Commonly used positively charged moieties include the amino acids lysine and arginine, although other positively charged moieties may also be useful. For example, lysine and arginine residues can be added to the carboxy or the N-terminus of a PNA strand.

Triplex-forming molecules may further be modified to be end capped to prevent degradation using a 3′ propylamine group. Procedures for 3′ or 5′ capping oligonucleotides are well known in the art.

Backbone modifications used to generate triplex-forming molecules should not prevent the molecules from binding with high specificity to the target site and inducing triplex formation

B. Triplex-Forming Target Sequence

The triplex-forming molecules bind to a predetermined target region referred to herein as the “target sequence”, “target region”, or “target site”. The target sequence for the triplex-forming molecules can be within or adjacent to a human gene encoding an encoding enzyme necessary for the metabolism of lipids, glycoproteins, or mucopolysaccharides in need of correction. The target sequence can be within the coding DNA sequence of the gene or within an intron. The target sequence can also be within DNA sequences which regulate expression of the target gene, including promoter or enhancer sequences.

The nucleotide sequences of the triplex-forming molecules are selected based on the sequence of the target sequence, the physical constraints, and the need to have a low dissociation constant (K_{d}) for the triplex-forming molecules/target sequence. As used herein, triplex-forming molecules are said to be substantially complementary to a target region when the triplex-forming molecules has a heterocyclic base composition which allows for the formation of a triple-helix with the target region. As such, a triplex-forming molecules is substantially complementary to a target region even when there are non-complementary bases present in the triplex-forming molecules.

There are a variety of structural motifs available which can be used to determine the nucleotide sequence of a substantially complementary oligonucleotide. Preferably, the triplex-forming molecules bind to or hybridize to the target sequence under conditions of high stringency and specificity. Reaction conditions for in vitro triple helix formation of an triplex-forming molecules probe or primer to a nucleic acid sequence vary from triplex-forming molecules to triplex-forming molecules, depending on factors such as the length triplex-forming molecules, the number of G:C and A:T base pairs, and the composition of the buffer utilized in the hybridization reaction.

1. Target Sequence Considerations for TFOs

Preferably, the TFO is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful. Most preferably, the oligonucleotides bind in a sequence-specific manner within the major groove of duplex DNA. An oligonucleotide substantially complementary, based on the third strand binding code, to the target region of the double-stranded nucleic acid molecule is preferred. The oligonucleotides will have a base composition which is conducive to triple-helix formation and will be generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C⁺.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

The oligonucleotides are preferably generated using known DNA synthesis procedures. In one embodiment, oligonucleotides are generated synthetically. Oligonucleotides can also be chemically modified using standard methods that are well known in the art.

2. Target Sequence Considerations for PNAs

Some triplex-forming molecules, such as PNA and tcPNA invade the target duplex, displacement of the polypyrimidine, and induce triplex formation with the displaced polypurine strand of the target duplex by both Watson-Crick and Hoogsteen binding. Preferably, both the Watson-Crick and Hoogsteen binding portions of the triplex forming molecules are substantially complementary to the target sequence. Although, as with triplex-forming oligonucleotides, a homopurine strand is needed to allow formation of a stable PNA/DNA/PNA triplex, PNA clamps can form at shorter homopurine sequences than those required by triplex-forming oligonucleotides and also do so with greater stability.

Preferably, PNAs are between 6 and 50 nucleotides in length. The Watson-Crick portion should be 9 or more nucleobases in length, optionally including a tail sequence. More preferably, the Watson-Crick binding portion is between about 9 and 30 nucleobases in length, optionally including a tail sequence of between 0 and about 15 nucleobases. More preferably, the Watson-Crick binding portion is between about 10 and 25 nucleobases in length, optionally including a tail sequence of between 0 and about 10 nucleobases. In the most preferred embodiment, the Watson-Crick binding portion is between 15 and 25 nucleobases in length, optionally including a tail sequence of between 5 and 10 nucleobases. The Hoogsteen binding portion should be 6 or more nucleobases in length. Most preferably, the Hoogsteen binding portion is between about 6 and 15 nucleobases, inclusive.

The triplex-forming molecules are designed to target the polypurine strand of a polypurine:polypyrimidine stretch in the target duplex nucleotide. Therefore, the base composition of the triplex-forming molecules may be homopyrimidine. Alternatively, the base composition may be polypyrimidine. The addition of a “tail” reduces the requirement for polypurine:polypyrimidine run. Adding additional nucleobases, known as a “tail,” to the Watson-Crick binding portion of the triplex-forming molecules allows the Watson-Crick binding portion to bind/hybridize to the target strand outside the site of strand displacement. These additional bases further reduce the requirement for the polypurine:polypyrimidine stretch in the target duplex and therefore increase the number of potential target sites. Triplex-forming oligonucleotides (TFOs) also require a polypurine:polypyrimidine to a form a triple helix. TFOs may require stretch of at least 15 and preferably 30 or more nucleotides. Peptide nucleic acids require fewer purines to a form a triple helix, although at least 10 or preferably more may be needed. Peptide nucleic acids including a tail, also referred to tail clamp PNAs, or tcPNAs, require even fewer purines to a form a triple helix. A triple helix may be formed with a target sequence containing fewer than 8 purines. Therefore, PNAs should be designed to target a site on duplex nucleic acid containing between 6-30 polypurine:polypyrimidines, preferably, 6-25 polypurine:polypyrimidines, more preferably 6-20 polypurine:polypyrimidines.

The addition of a “mixed-sequence” tail to the Watson-Crick-binding strand of the triplex-forming molecules such as PNAs also increases the length of the triplex-forming molecule and, correspondingly, the length of the binding site. This increases the target specificity and size of the lesion created at the target site and disrupts the helix in the duplex nucleic acid, while maintaining a low requirement for a stretch of polypurine:polypyrimidines. Increasing the length of the target sequence improves specificity for the target, for example, a target of 16 to 17 base pairs will statistically be unique in the human genome. Relative to a smaller lesion, it is likely that a larger triplex lesion with greater disruption of the underlying DNA duplex will be detected and processed more quickly and efficiently by the endogenous DNA repair machinery that facilitates recombination of the donor oligonucleotide.

The triple-forming molecules are preferably generated using known synthesis procedures. In one embodiment, triplex-forming molecules are generated synthetically. Triplex-forming molecules can also be chemically modified using standard methods that are well known in the art.

C. Methods for Determining Triplex Formation

A useful measure of triple helix formation is the equilibrium dissociation constant, K_{d}, of the triplex, which can be estimated as the concentration of triplex-forming molecules at which triplex formation is half-maximal. Preferably, the triplex-forming molecules have a binding affinity for the target sequence in the range of physiologic interactions. The preferred triplex-forming molecules have a K_{d} less than or equal to approximately 10⁻⁷ M. Most preferably, the K_{d} is less than or equal to 2×10⁻⁸ M in order to achieve significant intramolecular interactions. A variety of methods are available to determine the K_{d} of triplex-forming molecules with the target duplex. In the examples which follow, the K_{d} was estimated using a gel mobility shift assay (Durland et al., Biochemistry 30, 9246 (1991)). The dissociation constant (K_{d}) can be determined as the concentration of triplex-forming molecules in which half was bound to the target sequence and half was unbound.

D. Donor Oligonucleotides

The triplex forming molecules such as TFOs and PNAs may be administered in combination with, or tethered to, a donor oligonucleotide via a mixed sequence linker or used in conjunction with a non-tethered donor oligonucleotide that is substantially homologous to the target sequence. Triplex-forming molecules can induce recombination of a donor oligonucleotide sequence up to several hundred base pairs away. It is preferred that the donor oligonucleotide sequence is between 1 to 800 bases from the target binding site of the triplex-forming molecules. More preferably the donor oligonucleotide sequence is between 25 to 75 bases from the target binding site of the triplex-forming molecules. Most preferably that the donor oligonucleotide sequence is about 50 nucleotides from the target binding site of the triplex-forming molecules.

The donor sequence can contain one or more nucleic acid sequence alterations compared to the sequence of the region targeted for recombination, for example, a substitution, a deletion, or an insertion of one or more nucleotides. Successful recombination of the donor sequence results in a change of the sequence of the target region. Donor oligonucleotides are also referred to herein as donor fragments, donor nucleic acids, donor DNA, or donor DNA fragments. This strategy exploits the ability of a triplex to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered or unlinked fragments may range in length from 20 nucleotides to several thousand. The donor oligonucleotide molecules, whether linked or unlinked, can exist in single stranded or double stranded form. The donor fragment to be recombined can be linked or un-linked to the triplex forming molecules. The linked donor fragment may range in length from 4 nucleotides to 100 nucleotides, preferably from 4 to 80 nucleotides in length. However, the unlinked donor fragments have a much broader range, from 20 nucleotides to several thousand. In one embodiment the olignucleotide donor is between 25 and 80 nucleobases. In a further embodiment, the non-tethered donor nucleotide is about 50 to 60 nucleotides in length.

The donor oligonucleotides contain at least one mutated, inserted or deleted nucleotide relative to the target DNA sequence. Target sequences can be within the coding DNA sequence of the gene or within introns. Target sequences can also be within DNA sequences which regulate expression of the target gene, including promoter or enhancer sequences.

The donor oligonucleotides can contain a variety of mutations relative to the target sequence. Representative types of mutations include, but are not limited to, point mutations, deletions and insertions. Point mutations can cause missense or nonsense mutations. Deletions and insertions can result in frameshift mutations or deletions. These mutations may disrupt, reduce, stop, increase, improve, or otherwise alter the expression of the target gene. For example, it may be desirable to reduce or stop expression of an oncogene. Alternatively, it may be desirable to alter the polypeptide encoded by the target gene, for example, the human gene encoding α-L-iduronidase containing a non-sense point mutation that results in deficiency of the α-L-iduronidase enzyme.

Compositions including triplex-forming molecules may include one or more donor oligonucleotides. More than one donor oligonucleotides may be administered with triplex-forming molecules in a single transfection, or sequential transfections. Use of more than one donor oligonucleotide may be useful, for example, to create a heterozygous target gene where the two alleles contain different modifications.

Donor oligonucleotides are preferably DNA oligonucleotides, composed of the principal naturally-occurring nucleotides (uracil, thymine, cytosine, adenine and guanine) as the heterocyclic bases, deoxyribose as the sugar moiety, and phosphate ester linkages. Donor oligonucleotides may include modifications to nucleobases, sugar moieties, or backbone/linkages, as described above, depending on the desired structure of the replacement sequence at the site of recombination or to provide some resistance to degradation by nucleases. Modifications to the donor oligonucleotide should not prevent the donor oligonucleotide from successfully recombining at the recombination target sequence in the presence of triplex-forming molecules.

E. Methods for Determining Introduction of Alternative Sequence at the Target Site

As described in the example below, allele-specific PCR is a preferred method for determining if a recombination event has occurred. PCR primers are designed to distinguish between the original allele, and the new predicted sequence following recombination. Other methods of determining if a recombination event has occurred are known in the art and may be selected based on the type of modification made. Methods include, but are not limited to, analysis of genomic DNA, for example, by sequencing; analysis of mRNA transcribed from the target gene, for example, by Northern blot, in situ hybridization, real-time or quantitative reverse transcriptase (RT) PCT; and analysis of the polypeptide encoded by the target gene, for example, by immunostaining, ELISA, or FACS. In some cases, modified cells will be compared to parental controls. Other methods may include testing for changes in the function of the RNA transcribed by, or the polypeptide encoded by, the target gene.

If the target gene encodes an enzyme, an assay designed to test enzyme function or enzyme activity may be used. First, a standard curve is generated. For example, ratios of 2:98, 5:95, 10:90, 25:75, and 50:50 of cells wildtype and heterozygous for a mutation known to affect function of the enzyme would yield final WT allele frequencies of 1%, 2.5%, 5%, 12.5%, and 25% respectively. Protein can be isolated from a predetermined number of cells (i.e. 300,000-5000,000) of these mixed populations, as well as cells that are 100% homozygous wildtype, homozygous mutant, and heterozygous (+/−). Protein can be applied to an assay for enzyme function to determine the relative enzyme activity for the different cell populations, and data plotted on a line graph to generate standard curve. A suitable enzyme assay will be known to one of skill in the art and will depend on the enzyme of interest. For example, if the mutant gene/enzyme is α-L-iduronidase enzyme a suitable assay may the 4-methylumbelliferyl α-Iduronide (4MU) assay.

Once cells have been treated with triplex-forming molecules and donor oligonucleotides, the enzyme assay can be used identify cells exhibiting successful enzyme (gene) correction, or to measure the frequency of recombination in a treated population. For example, following treatment of cells with triplex-forming molecules and donor oligonucleotides, single cells can be isolated and expanded as individual clones. Total protein isolated from clonal populations can be applied to the enzyme assay and compared to the standard curve (prepared as described above) to identify clones exhibiting corrected enzyme function. Alternatively, total protein isolated from a treated (mixed) population can be applied to the enzyme assay and compared to the standard curve (prepared as described above) to estimate the number of cells (and % of total cells) from the treated population exhibiting corrected enzyme function or activity. This method is useful in determining the frequency of gene correction using different triplex-forming molecules, different donor oligonucleotides, and/or different amounts thereof. As described in the examples below, detection or measure of enzyme function or activity may be particularly useful for identifying cells that have been modified to correct dysfunction of a gene that contributes to a lysosomal storage disease.

F. Cell Targeting Moieties and Protein Transduction Domains

Formulations of the triplex-forming molecules embrace fusions of the triplex-forming molecules or modifications of the triplex-forming molecules, wherein the triplex-forming molecules are fused to another moiety or moieties. Such analogs may exhibit improved properties such as increased cell membrane permeability, activity and/or stability. Examples of moieties which may be linked or unlinked to the triplex-forming molecules, or donor oligonucleotides include, for example, targeting moieties which provide for the delivery of molecules or oligonucleotides to specific cells, e.g., antibodies to hematopoietic stem cells, CD34⁺ cells, T cells or any other preferred cell type, as well as receptor and ligands expressed on the preferred cell type. Preferably, the moieties target hematopoietic stem cells. Other moieties that may be provided with the triplex-forming molecules or oligonucleotides include protein transduction domains (PTDs), which are short basic peptide sequences present in many cellular and viral proteins that mediate translocation across cellular membranes. Exemplary protein transduction domains that are well-known in the art include the Antennapedia PTD and the TAT (transactivator of transcription) PTD, poly-arginine, poly-lysine or mixtures of arginine and lysine.

G. Additional Mutagenic Agents

The triplex-forming molecules can be used alone or in combination with other mutagenic agents. As used herein, two agents are said to be used in combination when the two agents are co-administered, or when the two agents are administered in a fashion so that both agents are present within the cell or blood simultaneously. In a preferred embodiment, the additional mutagenic agents are conjugated or linked to the triplex-forming molecule. Additional mutagenic agents that can be used in combination with triplex-forming molecules include agents that are capable of directing mutagenesis, nucleic acid crosslinkers, radioactive agents, or alkylating groups, or molecules that can recruit DNA-damaging cellular enzymes. Other suitable mutagenic agents include, but are not limited to, chemical mutagenic agents such as alkylating, bialkylating or intercalating agents. A preferred agent for co-administration is psoralen-linked molecules as described in PCT/US/94/07234 by Yale University.

H. Additional Prophylactic or Therapeutic Agents

The triplex-forming molecules can be used alone or in combination with other prophylactic or therapeutic agents. As used herein, two agents are said to be used in combination when the two agents are co-administered, or when the two agents are administered in a fashion so that both agents are present within the cell or serum simultaneously. Suitable additional prophylactic or therapeutic agents will be known to one of skill in the art and will depend on parameters such as the patient and condition to be treated.

It may also be desirable to administer compositions containing triplex-forming molecules in combination with agents that further enhance the frequency of gene correction in cells. For example, the compositions can be administered in combination with a histone deacetylase (HDAC) inhibitor, such as suberoylanilide hydroxamic acid (SAHA), which has been found to promote increased levels of gene targeting in asynchronous cells. The nucleotide excision repair pathway is also known to facilitate triplex-forming molecule-mediated recombination. Therefore, the compositions can be administered in combination with an agent that enhances or increases the nucleotide excision repair pathway, for example, an agent that increases the expression, activity, or localization to the target site, of the endogenous damage recognition factor XPA. Compositions may also be administered in combination with a second active agent that enhances uptake or delivery of the triplex-forming molecules or the donor oligonucleotides. For example, the lysosomotropic agent chloroquine has been shown to enhance delivery of PNAs into cells (Abes, et al., J. Controll. Rel., 110:595-604 (2006).

### II. Methods of Use

Triplex-forming molecules bind/hybridize to a target sequence within or adjacent to a human gene. The binding of the triplex-forming molecule to the target region stimulates mutations within or adjacent to the target region using cellular DNA synthesis, recombination, and repair mechanisms. The triplex-forming molecules can further be used to stimulate homologous recombination of an exogenously supplied, donor oligonucleotide, into a target region. Specifically, by activating cellular mechanisms involved in DNA synthesis, repair and recombination, the triplex-forming molecules can be used to increase the efficiency of targeted recombination. In targeted recombination, a triplex forming molecule is administered to a cell in combination with a separate donor oligonucleotide fragment which minimally contains a sequence substantially complementary to the target region or a region adjacent to the target region, referred to herein as the donor fragment. The donor fragment can further contain nucleic acid sequences which are to be inserted within the target region. The co-administration of a triplex forming molecules with the fragment to be recombined increases the frequency of insertion of the donor fragment within the target region when compared to procedures which do not employ a triplex forming molecules.

If the target gene contains a mutation that is the cause of a genetic disorder, then the oligonucleotide is useful for mutagenic repair that restores the DNA sequence of the target gene to normal. Alternatively, the oligonucleotide may induce a mutation into the wildtype target gene. Such modifications can be used to create new cell lines useful in studying lysosomal storage disease. Compositions containing triplex-forming molecules are also useful as a molecular biology research tool to cause targeted mutagenesis. Targeted mutagenesis has been shown to be a very useful tool when employed to not only elucidate functions of genes and gene products, but alter known activities of genes and gene products as well. Targeted mutagenesis of a specific gene in an animal oocyte, such as a mouse oocyte, provides a useful and powerful tool for genetic engineering for research and therapy and for generation of new strains of “transmutated” animals and plants for research and agriculture.

In targeted recombination, triplex forming molecules are administered to a cell in combination with a separate donor fragment which minimally contains a sequence essentially complementary to the target region or a region adjacent to the target region, referred to herein as the donor fragment. The triplex-forming molecules in conjunction with donor oligonucleotides can induce any of a range of mutations, including corrective mutations, in or adjacent to the target sequence. Representative types of mutations include, but are not limited to, point mutations, deletions and insertions. Point mutations can cause missense or nonsense mutations. As described in the examples below, recombination may also induce silent (i.e. synonymous mutations). Deletions and insertions can result in frameshift mutations or deletions. The donor fragment can differ from the target sequence at the one or more base positions that are desired to be substituted, inserted, deleted, or otherwise altered. In some embodiments, the donor fragment contains nucleic acid sequences which are to be inserted within the target region.

A. Methods of Use as a Molecular Research Tool

For in vitro research studies, a solution containing the triplex-forming molecules is added directly to a solution containing the DNA molecules of interest in accordance with methods well known to those skilled in the art and described in more detail in the examples below.

In vivo research studies are conducted by transfecting cells with the triplex-forming molecules and optionally one or more donor oligonuleotides in a solution such as growth media with the transfected cells for a sufficient amount of time for entry of the triplex-forming molecules into the cells for triplex formation with a target duplex sequence. Cells may transfected by electroporation or nucleofection, as described in the examples below, or through any other suitable means known in the art. The target duplex sequence may be chromosomal DNA, or episomal DNA, such as nonintegrated plasmid DNA. The target duplex sequence may also be exogenous DNA, such as plasmid DNA or DNA from a viral construct, which has been integrated into the cell's chromosomes. The target duplex sequence may also be a sequence endogenous to the cell. The transfected cells may be in suspension or in a monolayer attached to a solid phase, or may be cells within a tissue wherein the triplex-forming molecules are in the extracellular fluid.

B. Methods of Use for Treatment of Lysosomal Storage Diseases

Targeted DNA repair and recombination induced by triplex-forming molecules is especially useful to treat genetic deficiencies, disorders and diseases caused by mutations in single genes. Triplex-forming molecules are also especially useful to correct genetic deficiencies, disorders and diseases caused by point mutations. In preferred embodiments, the triplex-forming molecules in combination with one or more donor oligonucleotides induce site-specific mutations or alterations of the nucleic acid sequence within or adjacent to the target sequence within or is adjacent to a portion of human gene encoding a mutant enzyme that contributes to a lysosomal storage disease. Target sequences can be within the coding DNA sequence of the gene or within introns. Target sequences can also be within DNA sequences which regulate expression of the target gene, including promoter or enhancer sequences.

In one embodiment, compositions containing triplex-forming molecules and methods disclosed herein are employed to treat Gaucher's disease (GD). Gaucher's disease, also known as Gaucher syndrome, is the most common lysosomal storage disease. Gaucher's disease is an inherited genetic disease in which lipid accumulates in cells and certain organs due to deficiency of the enzyme glucocerebrosidase (also known as acid β-glucosidase) in lysosomes. Glucocerebrosidase enzyme contributes to the degradation of the fatty substance glucocerebroside (also known as glucosylceramide) by cleaving b-glycoside into b-glucose and ceramide subunits (Scriver C R, Beaudet A L, Valle D, Sly W S. The metabolic and molecular basis of inherited disease. 8th ed. New York: McGraw-Hill Pub, 2001: 3635-3668). When the enzyme is defective, the substance accumulates, particularly in cells of the mononuclear cell lineage, and organs and tissues including the spleen, liver, kidneys, lungs, brain and bone marrow.

There are two major forms: non-neuropathic (type 1, most commonly observed type in adulthood) and neuropathic (type 2 and 3). GBA (GBA glucosidase, beta, acid), the only known human gene responsible for glucosidase-mediated GD, is located on chromosome 1, location 1q21. More than 200 mutations have been defined within the known genomic sequence of this single gene (NCBI Reference Sequence: NG_{—}009783.1). The most commonly observed mutations are N370S, L444P, RecNcil, 84GG, R463C, recTL and 84 GG is a null mutation in which there is no capacity to synthesize enzyme. However, N370S mutation is almost always related with type 1 disease and milder forms of disease. Very rarely, deficiency of sphingolipid activator protein (Gaucher factor, SAP-2, saposin C) may result in GD. In some embodiments, triplex-forming molecules are used to induce recombination of donor oligonucleotides designed to correct mutations in GBA.

In another embodiment, triplex-forming molecules and the methods disclosed herein are used to treat Fabry disease (also known as Fabry's disease, Anderson-Fabry disease, angiokeratoma corporis diffusum and alpha-galactosidase A deficiency), a rare X-linked recessive disordered, resulting from a deficiency of the enzyme alpha galactosidase A (a-GAL A, encoded by GLA). The human gene encoding GLA has a known genomic sequence (NCBI Reference Sequence: NG_{—}007119.1) and is located at Xp22 of the X chromosome. Mutations in GLA result in accumulation of the glycolipid globotriaosylceramide (abbreviated as Gb3, GL-3, or ceramide trihexoside) within the blood vessels, other tissues, and organs, resulting in impairment of their proper function (Karen, et al., Dermatol. Online J 11 (4): 8 (2005)). The condition affects hemizygous males (i.e. all males), as well as homozygous, and potentially heterozygous (carrier), females. Males typically experience severe symptoms, while women can range from being asymptomatic to having severe symptoms. This variability is thought to be due to X-inactivation patterns during embryonic development of the female. In some embodiments, triplex-forming molecules are used to induce recombination of donor oligonucleotides designed to correct mutations in GLA.

In preferred embodiments, the disclosed compositions and methods are used to treat Hurler syndrome (HS). Hurler syndrome, also known as mucopolysaccharidosis type I (MPS I), α-L-iduronidase deficiency, and Hurler's disease, is a genetic disorder that results in the buildup of mucopolysaccharides due to a deficiency of α-L iduronidase, an enzyme responsible for the degradation of mucopolysaccharides in lysosomes (Dib and Pastories, Genet. Mol. Res., 6(3):667-74 (2007)). MPS I is divided into three subtypes based on severity of symptoms. All three types result from an absence of, or insufficient levels of, the enzyme α-L-iduronidase. MPS I H or Hurler syndrome is the most severe of the MPS I subtypes. The other two types are MPS I S or Scheie syndrome and MPS I H-S or Hurler-Scheie syndrome. Without α-L-iduronidase, heparan sulfate and dermatan sulfate, the main components of connective tissues, build-up in the body. Excessive amounts of glycosaminoglycans (GAGs) pass into the blood circulation and are stored throughout the body, with some excreted in the urine. Symptoms appear during childhood, and can include developmental delay as early as the first year of age. Patients usually reach a plateau in their development between the ages of two and four years, followed by progressive mental decline and loss of physical skills (Scott et al., Hum. Mutat. 6: 288-302 (1995)). Language may be limited due to hearing loss and an enlarged tongue, and eventually site impairment can results from clouding of cornea and retinal degeneration. Carpal tunnel syndrome (or similar compression of nerves elsewhere in the body) and restricted joint movement are also common.

The human gene encoding alpha-L-iduronidase (α-L-iduronidase; IDUA) is found on chromosome 4, location 4p16.3, and has a known genomic sequence (NCBI Reference Sequence: NG_{—}008103.1). Two of the most common mutations in IDUA contributing to Hurler syndrome are the Q70X and the W420X, non-sense point mutations found in exon 2 (nucleotide 774 of genomic DNA relative to first nucleotide of start codon) and exon 9 (nucleotide 15663 of genomic DNA relative to first nucleotide of start codon). of IDUA respectively. These mutations cause dysfunction alpha-L-iduronidase enzyme. As described in the examples below, two triplex-forming molecule target sequences including a polypurine:polypyrimidine stretches have been identified within the IDUA gene. One target site with the polypurine sequence 5′ CTGCTCGGAAGA 3′ (SEQ ID NO: 2) and the complementary polypyrimidine sequence 5′ TCTTCCGAGCAG 3′ (SEQ ID NO: 13) is located 170 base pairs downstream of the Q70X mutation. A second target site with the polypurine sequence 5′ CCTTCACCAAGGGGA 3′ (SEQ ID NO: 6) and the complementary polypyrimidine sequence 5′ TCCCCTTGGTGAAGG 3′ (SEQ ID NO: 14) is located 100 base pairs upstream of the W402X mutation. In preferred embodiments, triplex-forming molecules are designed to bind/hybridize in or near these target locations. In one preferred embodiment, a tcPNA with a sequence of Lys-Lys-Lys-JJT TJT-OOO-TCT TCC GAG CAG-Lys-Lys-Lys (SEQ ID NO: 1) binds to the target sequence downstream of the Q70X mutation. In another preferred embodiment a tcPNA with a sequence of Lys-Lys-Lys-T TJJ JJT-OOO-TCC CCT TGG TGA AGG -Lys-Lys-Lys (SEQ ID NO: 5) binds to the target sequence upstream of the W402X mutation. J=pseudoisocytosine and O=the flexible linker 8-amino-3,6-dioxaoctanoic acid monomers. Sequences are from N-terminus to C-terminus.

In the most preferred embodiments, triplex-forming molecules are administered according to the disclosed methods in combination with one or more donor oligonucleotides designed to correct the point mutations at Q70X or W402X mutations sites. In some embodiments, in addition to containing sequence designed to correct the point mutation at Q70X or W402X mutation, the donor oligonuclotides may also contain 7 to 10 additional, synonymous (silent) mutations. As described in the examples below, the additional silent mutations can facilitate detection of the corrected target sequence using allele-specific PCR of genomic DNA isolated from treated cells. In one preferred embodiment, the donor oligonucleotide with the sequence 5′ AGGACGGTCCCGGCCTGCGACACTTCCGCCCATAATTGTTCTTCATCT GCGGGGCGGGGGGGGG 3′ (SEQ ID NO: 15) is administered with triplex-forming molecules designed to target the binding site upstream of W402X to correct the W402X mutation in cells. In another preferred embodiment, the donor olignucleotide with the sequence 5′GGGACGGCGCCCACATAGGCCAAATTCAATTGCTGATCCCAGCTTA AGACGTACTGGTCAGCCTGGC 3′ (SEQ ID NO: 16) is administered with triplex-forming molecules designed to target the binding site downstream of Q70X to correct the of Q70X mutation in cells.

1. Ex Vivo Gene Therapy for Treating or Preventing Genetic Disorders

In one embodiment, ex vivo gene therapy of cells is used for the treatment of a genetic disorder in a subject. For ex vivo gene therapy, cells are isolated from a subject and contacted ex vivo with the compositions to produce cells containing mutations in or adjacent to genes. In a preferred embodiment, the cells are isolated from the subject to be treated or from a syngenic host. Target cells are removed from a subject prior to contacting with triplex-forming molecules and donor oligonucleotides. The cells can be hematopoietic progenitor or stem cells. In a preferred embodiment, the target cells are CD34⁺ hematopoietic stem cells. Hematopoietic stem cells (HSCs), such as CD34+ cells are multipotent stem cells that give rise to all the blood cell types including erythrocytes. Therefore, CD34+ cells can be isolated from a patient with lysosomal storage disease, the mutant gene altered or repaired ex-vivo using the disclosed compositions and methods, and the cells reintroduced back into the patient as a treatment or a cure.

Stem cells can be isolated and enriched by one of skill in the art. Methods for such isolation and enrichment of CD34⁺ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

In humans, CD34⁺ cells can be recovered from cord blood, bone marrow or from blood after cytokine mobilization effected by injecting the donor with hematopoietic growth factors such as granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cells can be selected by positive and negative selection techniques. Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. Other techniques involve the use of fluorescence activated cell sorting (FACS). The CD34 antigen, which is found on progenitor cells within the hematopoietic system of non-leukemic individuals, is expressed on a population of cells recognized by the monoclonal antibody My-10 (i.e., express the CD34 antigen) and can be used to isolate stem cell for bone marrow transplantation. My-10 deposited with the American Type Culture Collection (Rockville, Md.) as HB-8483 is commercially available as anti-HPCA 1. Additionally, negative selection of differentiated and “dedicated” cells from human bone marrow can be utilized, to select against substantially any desired cell marker. For example, progenitor or stem cells, most preferably CD34⁺ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium comprising cell surface factors supporting the proliferation of stem cells. Stromal cells may be freed of hematopoietic cells employing appropriate monoclonal antibodies for removal of the undesired cells.

The isolated cells are contacted ex vivo with a combination of triplex-forming molecules and donor oligonucleotides in amounts effective to cause the desired mutations in or adjacent to genes in need of repair or alteration, for example the human α-L-iduronidase gene. These cells are referred to herein as modified cells. Methods for transfection of cells with oligonucleotides and peptide nucleic acids are well known in the art (Koppelhus, et al., Adv. Drug Deliv. Rev., 55(2): 267-280 (2003)). It may be desirable to synchronize the cells in S-phase to further increase the frequency of gene correction. Methods for synchronizing cultured cells, for example, by double thymidine block, are known in the art (Zielke, et al., Methods Cell Biol., 8:107-121 (1974)).

The modified cells can be maintained or expanded in culture prior to administration to a subject. Culture conditions are generally known in the art depending on the cell type. Conditions for the maintenance of CD34⁺ in particular have been well studied, and several suitable methods are available. A common approach to ex vivo multi-potential hematopoietic cell expansion is to culture purified progenitor or stem cells in the presence of early-acting cytokines such as interleukin-3 It has also been shown that inclusion, in a nutritive medium for maintaining hematopoietic progenitor cells ex vivo, of a combination of thrombopoietin (TPO), stem cell factor (SCF), and flt3 ligand (Flt-3L; i.e., the ligand of the flt3 gene product) was useful for expanding primitive (i.e., relatively non-differentiated) human hematopoietic progenitor cells in vitro, and that those cells were capable of engraftment in SCID-hu mice (Luens et al., 1998, Blood 91:1206-1215). In other known methods, cells can be maintained ex vivo in a nutritive medium (e.g., for minutes, hours, or 3, 6, 9, 13, or more days) comprising murine prolactin-like protein E (mPLP-E) or murine prolactin-like protein F (mPIP-F; collectively mPLP-E/IF) (U.S. Pat. No. 6,261,841). It will be appreciated that other suitable cell culture and expansion method can be used in accordance with the invention as well. Cells can also be grown in serum-free medium, as described in U.S. Pat. No. 5,945,337.

In another embodiment, the modified hematopoietic stem cells are differentiated ex vivo into CD4⁺ cells culture using specific combinations of interleukins and growth factors prior to administration to a subject using methods well known in the art. The cells may be expanded ex vivo in large numbers, preferably at least a 5-fold, more preferably at least a 10-fold and even more preferably at least a 20-fold expansion of cells compared to the original population of isolated hematopoietic stem cells.

In another embodiment cells for ex vivo gene therapy, the cells to be used can be dedifferentiated somatic cells. Somatic cells can be reprogrammed to become pluripotent stem-like cells that can be induced to become hematopoietic progenitor cells. The hematopoietic progenitor cells can then be treated with triplex-forming molecules and donor oligonucleotides as described above with respect to CD34⁺ cells to produce recombinant cells having one or more modified genes. Representative somatic cells that can be reprogrammed include, but are not limited to fibroblasts, adipocytes, and muscles cells. Hematopoietic progenitor cells from induced stem-like cells have been successfully developed in the mouse (Hanna, J. et al. Science, 318:1920-1923 (2007)).

To produce hematopoietic progenitor cells from induced stem-like cells, somatic cells are harvested from a host. In a preferred embodiment, the somatic cells are autologous fibroblasts. The cells are cultured and transduced with vectors encoding Oct4, Sox2, Klf4, and c-Myc transcription factors. The transduced cells are cultured and screened for embryonic stem cell (ES) morphology and ES cell markers including, but not limited to AP, SSEA1, and Nanog. The transduced ES cells are cultured and induced to produce induced stem-like cells. Cells are then screened for CD41 and c-kit markers (early hematopoietic progenitor markers) as well as markers for myeloid and erythroid differentiation.

The modified hematopoietic stem cells or modified induced hematopoietic progenitor cells are then introduced into a subject. Delivery of the cells may be effected using various methods and includes most preferably intravenous administration by infusion as well as direct depot injection into periosteal, bone marrow and/or subcutaneous sites.

The subject receiving the modified cells may be treated for bone marrow conditioning to enhance engraftment of the cells. The recipient may be treated to enhance engraftment, using a radiation or chemotherapeutic treatment prior to the administration of the cells. Upon administration, the cells will generally require a period of time to engraft. Achieving significant engraftment of hematopoietic stem or progenitor cells typically takes weeks to months.

A high percentage of engraftment of modified hematopoietic stem cells is not envisioned to be necessary to achieve significant prophylactic or therapeutic effect. It is expected that the engrafted cells will expand over time following engraftment to increase the percentage of modified cells. In some embodiments, the modified cells have a corrected α-L-iduronidase gene. Therefore, in a subject with Hurler syndrome, the modified cells are expected to improve or cure the condition. It is expected that engraftment of only a small number or small percentage of modified hematopoietic stem cells will be required to provide a prophylactic or therapeutic effect.

In preferred embodiments, the cells to be administered to a subject will be autologous, e.g. derived from the subject, or syngenic. Nevertheless, allogeneic cell transplants are also envisioned, and allogeneic bone marrow transplants are carried out routinely. Allogeneic cell transplantation can be offered to those patients who lack an appropriate sibling donor by using bone marrow from antigenically matched, genetically unrelated donors (identified through a national registry), or by using hematopoietic progenitor or stem-cells obtained or derived from a genetically related sibling or parent whose transplantation antigens differ by one to three of six human leukocyte antigens from those of the patient.

2. In Vivo Gene Therapy

In another embodiment, the triplex-forming molecules are administered directly to a subject in need of gene alteration.

a. Formulations

The disclosed compositions including triplex-forming molecules and one or more donor fragments are preferably employed for therapeutic uses in combination with a suitable pharmaceutical carrier. Such compositions include an effective amount of triplex-forming molecules and donor fragment, and a pharmaceutically acceptable carrier or excipient. An effective amount of triplex-forming molecules may be enough molecules to induce formation of a triple helix at the target site. An effective amount of triplex-forming molecules may also be an amount effective to increase the rate of recombination of a donor fragment relative to administration of the donor fragment in the absence of triplex-forming molecules. Compositions should include an amount of donor fragment effective to recombine at the target site in the presence of triplex-forming molecules. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids.

It is understood by one of ordinary skill in the art that nucleotides administered in vivo are taken up and distributed to cells and tissues (Huang, et al., FEBS Lett., 558(1-3):69-73 (2004)). For example, Nyce, et al. have shown that antisense oligodeoxynucleotides (ODNs) when inhaled bind to endogenous surfactant (a lipid produced by lung cells) and are taken up by lung cells without a need for additional carrier lipids (Nyce, et al., Nature, 385:721-725 (1997)). Small nucleic acids are readily taken up into T24 bladder carcinoma tissue culture cells (Ma, et al., Antisense Nucleic Acid Drug Dev., 8:415-426 (1998)).

The disclosed compositions including triplex-forming molecules, such as TFOs and PNAs, and donor fragments may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example, in Sambrook et al., Molecular Cloning: A Laboratory Manual, Cold Spring Harbor Laboratory, New York (1989); and Ausubel et al., Current Protocols in Molecular Biology, John Wiley & Sons, New York (1994). Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The triplex-forming molecules alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the compositions including triplex-forming molecules and donor oligonucleotides described above may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the triplex-forming molecules and/or donor oligonucleotides are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett., 14(19):4975-4977 (2004)) and in vivo (Soutschek, et al., Nature, 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol., 59(11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

b. Methods of Administration

In general, methods of administering compounds, including oligonucleotides and related molecules, are well known in the art. In particular, the routes of administration already in use for nucleic acid therapeutics, along with formulations in current use, provide preferred routes of administration and formulation for the triplex-forming molecules described above. Preferably the triplex-forming molecules and donor oligonucleotides are injected into the organism undergoing genetic manipulation, such as an animal requiring gene therapy or anti-viral therapeutics.

The disclosed compositions including triplex-forming molecules and donor oligonucleotides can be administered by a number of routes including, but not limited to, oral, intravenous, intraperitoneal, intramuscular, transdermal, subcutaneous, topical, sublingual, or rectal means. The preferred route of administration is intravenous. Triplex-forming molecules and oligonucleotides can also be administered via liposomes. Such administration routes and appropriate formulations are generally known to those of skill in the art.

Administration of the formulations may be accomplished by any acceptable method which allows the triplex-forming molecules and a donor nucleotide, to reach their targets.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The triplex-forming molecules and donor oligonucleotide may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the triplex-forming molecules, and donor oligonucleotides, over a certain time period, for example, hours, days, weeks, months or years. This may be accomplished, for example, by repeated administrations of a formulation or by a sustained or controlled release delivery system in which the compositions are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the oligonucleotides are contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the triplex-forming molecules and donor oligonucleotides. In addition, a pump-based hardware delivery system may be used to deliver one or more embodiments.

Examples of systems in which release occurs in bursts include systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Compositions including triplex-forming molecules and donor oligonucleotides and methods of their use will be further understood in view of the following non-limiting example.

### EXAMPLES

### Example 1

### Triplex Formation at Two IDUA Gene Target

Materials and Methods

Design of Triplex-Forming Molecules

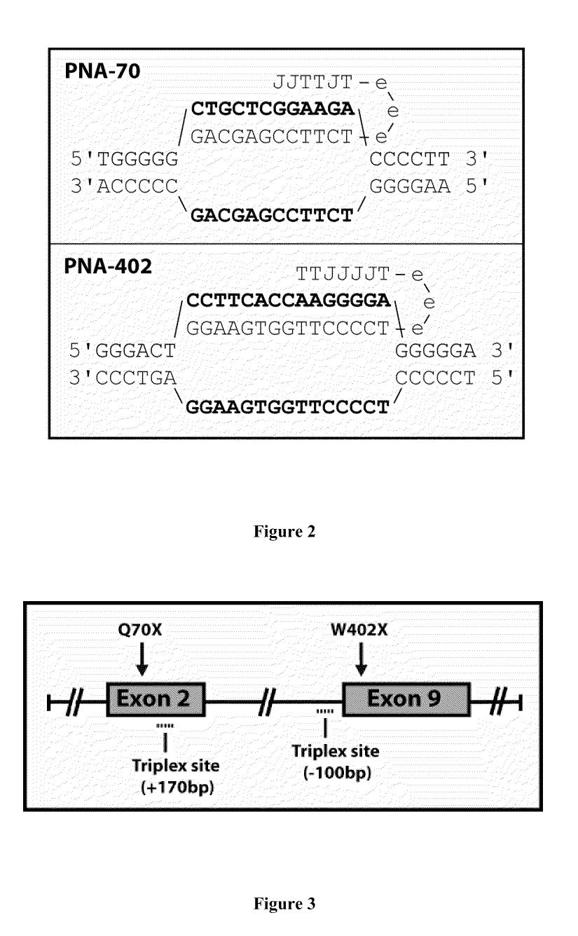

The generic sequences for IDUA402tc715 and IDUA7Otc612 are depicted schematically in FIG. 2. IDUA402tc715 is a tail clamp peptide nucleic acid (tcPNA) with the sequence Lys-Lys-Lys-TTJJJJT-OOO-TCCCCTTGGTGAAGG-Lys-Lys-Lys (SEQ ID NO: 5). This triplex-forming molecule contains a 7 base pair Hoogsteen binding portion and 15 base pair Watson-Crick binding portion, where 7 bases of the Watson-Crick binding portion contribute to PNA:DNA:PNA triplex formation, and the “tail” end 8 bases contribute to PNA:DNA duplex formation. IDUA7Otc612 is a tail clamp peptide nucleic acid (tcPNA) with Lys-Lys-Lys-JJTTJT-OOO-TCTTCCGAGCAG-Lys-Lys-Lys (SEQ ID NO: 1). This triplex-forming molecule contains a 6 base pair Hoogsteen binding portion and 12 base pair Watson-Crick binding portion, where 6 bases of the Watson-Crick binding portion contribute to PNA:DNA:PNA triplex formation, and the tail 6 bases contribute to PNA:DNA duplex formation, J=pseudoisocytosine and O=flexible 8-amino-3,6-dioxaoctanoic acid, 6-aminohexanoic acid monomers. Both ends of each tail clamp PNA are capped with three lysines (Lys). PNA were cleaned-up and purified using an Ambion Ultra Filter MWCO 3k.

Cloning PNA Targeting Plasmids

PNA-70 Binding Plasmid pHT5:

Site directed mutagenesis was performed to generate the PNA-70 binding site in pcDNA5/FRT using the Quickchange site directed mutagenensis kit (Stratagene). Primers used were complementary to each other,

70PNASDMF
(SEQ ID NO: 17)
5′GACAGCAAGGGGGAGGATTGCTG
CTCGGAAGACAATAGCAGGCATG3′
and
70PNASDMR
(SEQ ID NO: 18)
5′ CATGCCTGCTATTGTCTTCCGAGC
AGCAATCCTCCCCCTTGCTGTC 3′.

Reactions were run with the following protocol. 95° C. 30 seconds, (95° C. 30 seconds, 55° C. 1 minute, 68° C. 5 minutes 30 seconds)×18 cycles. Reactions were digested with DpnI to remove parental template plasmid and transformed into TOP10 chemically competent bacteria and plated on LB plates supplemented with 100 ug/mL ampicillin and grown overnight at 37° C. Individual colonies picked and grown overnight. Subsequently plasmid DNA was prepped using Qiagen miniprep kit.
PNA-402 Binding Plasmid pHT6:

A short 324 by fragment containing the PNA402 binding site of the IDUA gene was amplified from gDNA harvested from K562 cells using Pfx Taq polymerase (Invitrogen). PCR reactions were supplemented with 1 × Enhancer (Invitrogen) and 1M Betaine (Sigma) due to a high G/C content. PCR conditions were as follows:
94° C. 2 minutes,
(94° C. 15 seconds, 50.8° C. 30 seconds, 68° C. 30 seconds)×45cycles
68° C. 1 minute. Primers used were

PNA402BAM
(SEQ ID NO: 19)
5′ CGGTGCGGATCCGCTGCGGGGAGCGCACTTC
and
PNA402R
(SEQ ID NO: 20)
5′ GTGTCGTCGCTCGCGTAG.

The PCR reactions was directly digested with BamHI and ApaI and gel purified using the Qiagen Gel Extractions kit. pcDNA5/FRT was also cut with BamHI and ApaI, and gel purified. These two gel pure fragments were ligated together with T4 DNA ligase (New England Biolabs) in a ratio of 3 molar PCR fragment: 1 molar pcDNA5 vector for 30 minutes at room temperature, and transformed into DH5α competent bacteria and plated on LB plates supplemented with 100 ug/mL ampicillin and grown overnight at 37° C. Colonies were grown and plasmid DNA was prepped (pHT6) using Qiagen plasmid extraction kit and sequenced.

Gel Mobility Shift Assays

PNA-70 targeting Q70 was incubated at various concentrations with 1.5 μg pHT5 plasmid DNA, 10 mM KCl, and Tris-EDTA (TE) overnight at 37° C., These reactions were then digested with restriction enzymes Xho and SphI for 2 hrs at 37° C. Similarly, PNA-402 targeting the W402 locus was incubated with 1.5 μtg pHT6 plasmid at various concentrations and digested BamHI and ApaI. Reactions were run on an 8% native bis-acylamide gel and silver stained, and imaged on a G:Box gel doc (Syngene)

Results

The Q70X and W402X IDUA gene mutations are found in up to 70% of Caucasian Hurler Syndrome (HS) patients. The non-sense point mutations result in premature stop codons that cause IDUA enzyme deficiency in patients. Triplex binding sites were identified near each of these mutations (FIG. 3). These sequences were deemed potentially useful as PNA or TFO binding targets, with the intention of stimulating recombination, since triplex-induced recombination can occur over distances of up to several hundred bps. Two separate tail-clamp PNAs (tcPNAs) that bind these sites, IDUA7Otc612 (PNA-70) and IDUA402tc715 (PNA-402) having the generic sequences JJTTJT-EEE-TCTTCCGAGCAG (SEQ ID NO: 29) and TTJJJJT-EEE-TCCCCTTGGTGAAGG (SEQ ID NO: 30), respectively were made (where J=pseudoisocytosine and E=flexible linker). These tcPNAs contained two linked PNA segments and were designed to form a PNA/DNA/PNA triplex clamp on the purine-rich DNA strand of the site. The mixed base extension of the Watson-Crick polypyrimidine strand increases the specificity of the binding reaction. Gel mobility shift assays were performed to test the affinity of PNA-70 and PNA-402 to their respective binding sites in the IDUA gene at increasing concentration (0, 0.2 μM, 0.4 μM, 0.8 μM, 1.2 μM) in vitro (FIGS. 10 and 11). Incubation of both PNA-70 and PNA-402 with target duplex DNA resulted in band shift indicating the formation of triplex. Strong binding was observed by both molecules to their corresponding targets. These data indicate that PNA-70 and PNA-402 are viable triplex-forming molecules to induce targeted recombination of the IDUA gene.

### Example 2

### Targeted Modification of the IDUA Gene

Materials and Methods

Cell Lines

Human CD34 stem cells (Lonza), K562, THP-1, human primary fibroblasts (Coriell cell repository).

Cell Media

CD34 cell medium (Stemspan with cc110 cytokine cocktail (Stemcell technologies)), THP/K562 culture medium (RPMI 1640, 10% FBS, 1% L-glu, 1% P/S), Fibroblast culture medium (DMEM, 10-15% FBS, 1% L-glu, 1% P/S).

Donor Oligonucleotides

W402XCM is a single stranded donor DNA oligonucelotide with the sequence

(SEQ ID NO: 15)
5′AGGACGGTCCCGGCCTGCGACACTTCCGCCCAT
AATTGTTCTTCATCTGCGGGGCGGGGGGGGG3′.

Q70XCM is a single stranded donor DNA oligonucleotide with the sequence

(SEQ ID NO: 16)
5′GGGACGGCGCCCACATAGGCCAAATTCAATTGCT
GATCCCAGCTTAAGACGTACTGGTCAGCCTGGC3′.

Each donor contains phosphothioate linkages at first 3 and last 3 bases.

Transfection Equipment

CD34 and primary fibroblasts were transfected by nucleofection: Amaxa Nucleofector, CD34 nucleofector kit; or Primary Fibroblast nucleofector kit. THP1 and K562 cells were transfected by square wave electroporation using 0.4 cm cuvettes in a BioRad Genepulser MxCell.

Allele Specific PCR

Genomic DNA was extracted from treated cells using SV genomic DNA extraction kit (Promega). DNA from each sample was quantitated and diluted to 45 ng/μl. Two step allele specific PCR was performed on genomic DNA, where the first PCR amplified the region flanking the allele and the second PCR amplified only the codon modified allele. PCR results were resolved on a 2% agarose gel (Nuseive 3:1). PCR reagents included Platinum Taq (Invitrogen), dNTP's, 50 mM MgCl2, 5M Betaine, 10×PCR×Enhancer solution, filtered pipette tips, gel casting system (Galileo), power supply (BioRad), 1× TBE, 100 bp ladder (Invitrogen). IDUA specific primers included:

402XS*
(SEQ ID NO: 21)
5′ TGGCGGGGCCTGGGGACTCCTTCACCAA 3′
402XAS*
(SEQ ID NO: 22)
5′ GCGGGTGTCGTCGCTCGCGTAGAT 3′
IDUA402CM
(SEQ ID NO: 23)
5′ GAAGAACAATTATGGGCGGAAGT 3′
W402X100R
(SEQ ID NO: 24)
5′ CCTGGGGGCGGTGGGCGCTG 3′
Q70XS*
(SEQ ID NO: 25)
5′ CGCTGCCAGCCATGCTGAGGCTCG 3′
Q70XAS*
(SEQ ID NO: 26)
5′ ACACAGGGATGCTCACGGGTGCAC 3′
IDUA70CMF
(SEQ ID NO: 27)
5′ TTAAGCTGGGATCAGCAATTGAATTTG 3′
IDUA70ASR
(SEQ ID NO: 28)
5′ ACAGCCAGCAAGGACACGCTC 3′
(Beesley et. al Human Genet 109 (2001))

Results

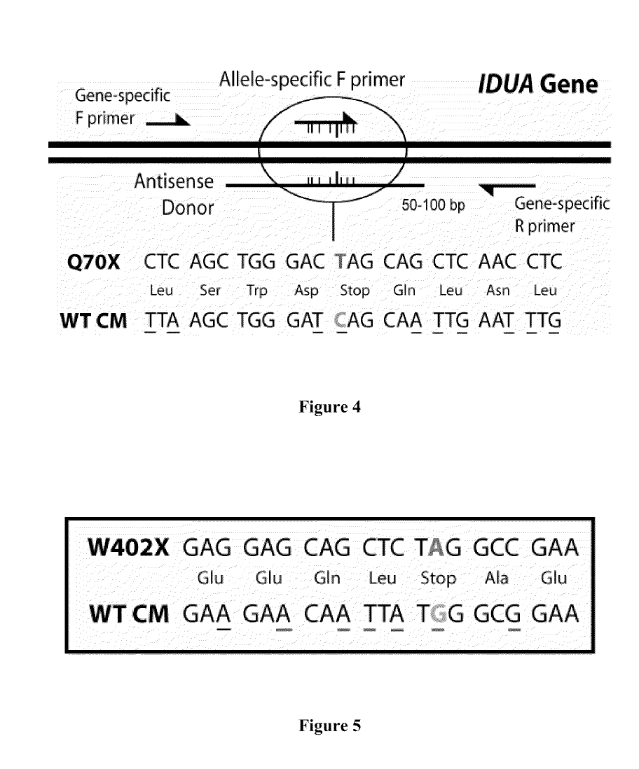

Next PNA-70 and PNA-402 in combination with a donor oligonucleotide were tested for the ability to induce targeted modification of the endogenous IDUA gene. For these studies, an allele-specific (AS) PCR approach was developed to detect modifications in the endogenous IDUA gene. An AS (forward) primer was designed with bases complementary to the base substitutions being detected, along with a gene-specific (reverse) primer located 200 bps downstream from the modification. Amplification of the modified allele is favored because of significantly impaired annealing and extension on the WT versus the modified allele, especially when the AS primer contains a modified by at the 3′-terminus. A schematic of the AS-PCR approach is shown in FIG. 4. The AS-PCR assay was designed to detect a cluster of synonymous, single base substitutions, located at the Q70X and W402X mutation sites, which do not change the WT amino acid sequence of the encoded IDUA protein. A total of 10 and 7 base substitutions were designed for introduction into the IDUA locus at the Q70X and the W420X mutation sites, respectively, which are referred to as WT codon modified or WT CM (shown in FIGS. 4 and 5). Optimization of PCR amplification conditions was performed using plasmids containing WT and the corresponding WT CM IDUA fragments.

PNA-70 and PNA-402 and the corresponding WT CM donor DNAs were tested for their ability to modify the endogenous IDUA gene in K562 cells, THP-1 cells, normal human fibroblasts and human CD34+ cells (FIGS. 4 and 5). The neutral peptide backbone of PNAs limits the use of cationic lipids as a delivery reagent for these molecules. Thus, K562 cells and primary fibroblasts were transfected via exponential decay electroporation. Nucleofection was used for the transfection of PNA/DNA molecules into human CD34+ cells from an apheresis collection of peripheral blood stem cells mobilized by G-CSF in healthy donors and selected for CD34 using the Baxter Isolex (Deerfield, Ill.). Next, cells were transfected with an end-protected antisense 70 bp single stranded DNA donor, containing one of the two WT CM modifications, either alone or in combination with the corresponding PNA. After 48 h, cells were harvested and genomic DNA was prepared for PCR analysis, using the AS primers. Allele-specific PCR revealed a high level of PNA-induced IDUA gene modification following treatment with PNA-70 and WT CM donor DNA in THP-1 cells, compared to donor alone. Similar results were observed in K562 cells. Similar levels of PNA-induced IDUA gene modification at the W402X mutation site were also observed in K562 cells, normal human fibroblasts and CD34+ cells (FIGS. 12 and 13). PCR analysis, using gene-specific primers, confirmed equal amounts of starting genomic DNA template for each sample. These data show that PNAs can be combined with donor DNA to modify the endogenous IDUA gene locus in human cells, including CD34+ cells.

### Example 3

### Partial Restoration of IDUA Enzyme Activity Following 402CM Donor/402-tc715 PNA Treatment of Hurler Primary Fibroblasts

Materials and Methods

4MU Standard Curve

4-methylumbelliferyl α-Iduronide (4MU) is a naturally fluorescent compound which can be analyzed using a fluorimeter with a UV wavelength. A standard curve is necessary to determine the amount of 4MU released from 4MUI substrate when acted on by functional IDUA enzyme. Materials and reagents required for standard curve include: 4MU (MP-cat #152475) sodium salt M.W.=198.2: Stock A—1 mM and Stock B—1 uM diluted in deionized distilled water (4MU is a light sensitive reagent and should be stored at 4° C. when it is not in use); Stop Buffer (0.5M Glycine-0.2M Carbonate-pH=10.2; 100 ul Cuvettes (Turner Biosystems, P/N 7000-950); Fluorimeter (Turner Biosystems); deionized distilled water. Dilutions were made in quadruplicate according to chart 1 (below) in deionized distilled water.

CHART 1
Dilutions for 4MU Standard Curve

| | | | Amt. | | |
|---|---|---|---|---|---|
| | | New | added to | | Pmoles |
| Solution | Dilution | conc. | minicell* | Final conc. | added |
| Stock A | 1:250 | 4 uM | 5 ul | 200 nM | 20 |
| (1 mM) | | | | | |
| | 1:500 | 2 uM | 5 ul | 100 nM | 10 |
| | 1:2 (of 1:500 | 1 uM | 5 ul | 50 nM | 5 |
| | above) | | | | |
| Stock B | 1:5 | 200 nM | 5 ul | 10 nM | 1 |
| (1 uM) | | | | | |
| | 1:50 | 20 nM | 5 ul | 1 nM | 0.1 |
| DDH20 | N/A | N/A | 5 ul | N/A | N/A |

*Minicell contains 95 μl stop buffer.
Tubes were vortexed well, covered with foil, and incubated at room temperature for 10 minutes. After incubation, samples were vortex again and 5 μl was added to a cuvette containing the 95 ul of stop buffer. Samples were pipeted up and down 10 times to ensure proper mixing. Fluorescence was detected on fluorimeter (UV, units FSU).

Collection of IDUA Enzyme from Treated Cells

To measure cellular enzyme activity 4MUI was used as a substrate. 4MUI is not fluorescent until acted upon by a functional IDUA enzyme. 100 ng/ml of recombinant human IDUA (rhIDUA) was used as a positive control. Between 300,000 and 500,000 cells per cell line were collected by centrifugation and washed 1× with PBS. The media was discarded, the remaining cell pellet was resuspended in 50 μl Lysis buffer (0.9% NaCl, 0.2% Triton® X-100 in water-pH 3.5), and subjected to 3 freeze-thaw cycles using dry ice and a water bath set to 37° C. Insoluble material was removed by centrifugation for 13,000 g for 5 minutes. Supernatant was retained, and protein content was quantified. 25 μl of the lysed sample was incubated with 25 μl of 200 μM 4-methylumbelliferyl α-Iduronide substrate at room temperature for 1 hour, and reaction was stopped by adding 500 μl of 0.5M glycine-0.2M carbonate buffer pH 10.2. Samples were mix thoroughly and fluorescence was detected on a fluorimeter.

Generation of Standard Curve, Plotting Enzyme Function vs. Allele Frequency

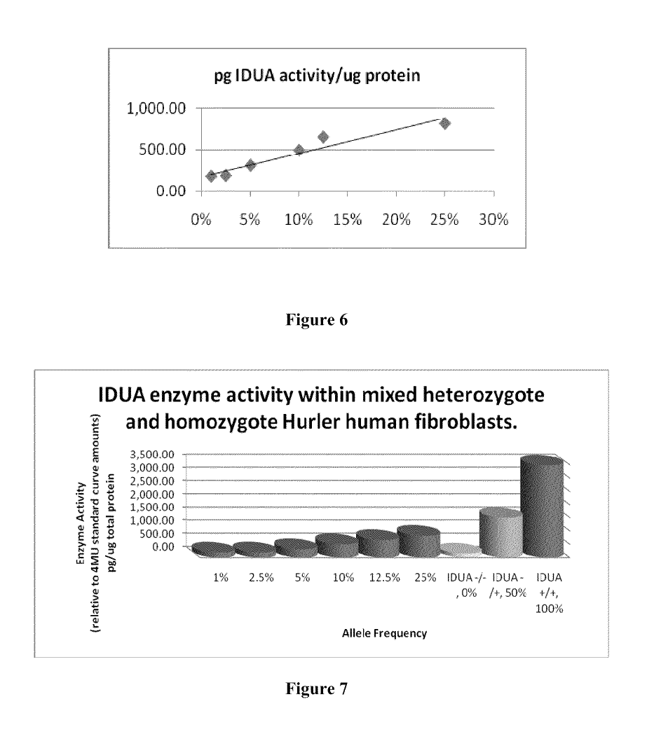

Heterozygous W402X+/−human primary fibroblasts were mixed with homozygous W402−/−fibroblasts at ratios of 2:98, 5:95, 10:90, 25:75, and 50:50, giving final WT allele frequencies of 1%, 2.5%, 5%, 12.5%, and 25% respectively. Enzyme activity was measured using 4MUI as a substrate and referenced to a 4MU standard curve to identify the amount of fluorescence generated by enzymatic activity (FIG. 6).

Nucleofection of Donor/PNA into Hurler Fibroblasts

1×10⁶ Hurler fibroblasts were nucleofected with 4 μM or 6 μM W402CM donor and 4 uμM W402-tc715 PNA using the primary fibroblast kit from Lonza. The pre-set program V-013 was used on a Amaxa nucleofector machine. 48 hrs later the cells were assayed for enzyme activity.

Results

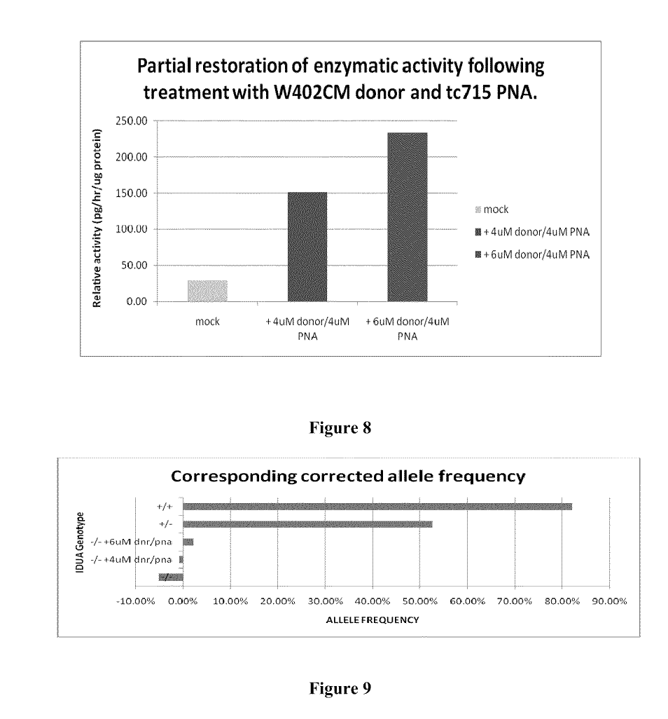

Human primary fibroblasts homozygous for the IDUA mutation W402X (“Hurler cells”) associated with a severe form of Hurler disease were electroporated with 4 μM W402CM donor or 6 μM W402 donor along with 4 μM W402-tc715 PNA (also called IDUA402tc715, also called PNA-402). The W402CM donor was designed to repair the stop codon (X) back to tryptophan (W) and restore functional enzyme capability. To test this, a fluorescent enzyme functional assay was performed using 4MUI as a substrate. Fluorescent values were normalized to total protein and were referenced against a standard curve generated by mixing various ratios of heterozygous primary fibroblasts with homozygous mutant fibroblasts and plotting enzyme function against allele frequency (FIGS. 6 and 7). There was a significant increase in enzymatic function in samples treated with donor and PNA when compared to Hurler cells which have background levels of fluorescence. Moreover a dose response was discovered, as 6 μM of donor boosted enzyme function more than 4 μM (FIG. 8). Using the standard curve, the data was extrapolated. In this way it was estimated that close to 2% of cells treated with 6 μM donor/4 μM PNA exhibited repair as evidenced by restored enzyme function (FIG. 9). It is believed that corrected allele frequencies above 1% would likely show clinical benefit due to the propensity of cross correction from corrected cells over to un-repaired mutant cells.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A recombinagenic or mutagenic composition comprising a donor oligonucleotide and a single-stranded triplex-forming molecule having a sequence that forms a triple-stranded nucleic acid molecule with a target sequence double-stranded nucleic acid molecule,
wherein the target sequence is composed of a stretch of polypurines or polypyrimidines located between 1 and 800 nucleotides from the target sequence of the donor oligonucleotide, and
wherein the target sequence of the donor oligonucleotide is within or adjacent to a human gene encoding an enzyme necessary for the metabolism of lipids, glycoproteins, or mucopolysaccharides
wherein the target sequence of the donor olignucleotide contains one or more mutations in need of correction.

2. The recombinagenic or mutagenic composition of claim 1 wherein the donor fragment is between 4 and 100 nucleotides in length, more preferably between 25 and 80 nucleobases.

3. The recombinagenic or mutagenic composition of claim 1 wherein the donor fragment is linked to the triplex-forming composition.

4. The recombinagenic or mutagenic composition of claim 1 wherein the triplex-forming molecule is selected from the group consisting of a triplex-forming oligonucleotide and a peptide nucleic acid.

5. The recombinagenic or mutagenic composition of claim 4 wherein the peptide nucleic acid is two peptide nucleic acids linked by a flexible linker such that the peptide nucleic acid forms a clamp at the duplex DNA target site.

6. The recombinagenic or mutagenic composition of claim 5 wherein the Watson-Crick binding portion is between about 9 and 30 nucleobases in length, including a tail sequence of up to 15 nucleobases.

7. The recombinagenic or mutagenic composition of claim 1 wherein the target sequence of the donor oligonucleotide is within or adjacent to a gene selected from the group consisting of GBA, GLA, and α-L-iduronidase.

8. The recombinagenic or mutagenic composition of claim 7 wherein the target sequence of the donor oligonucelotide contains a point mutation.

9. The recombinagenic or mutagenic composition of claim 1 wherein the target sequence of the donor oligonucleotide is within or adjacent the α-L-iduronidase gene containing W402X or Q70X mutations.

10. The recombinagenic or mutagenic composition of claim 1 wherein the target sequence of the triplex-forming molecule contains part or all of the sequence 5′ CTGCTCGGAAGA 3′ (SEQ ID NO: 2).

11. The recombinagenic or mutagenic composition of claim 1 wherein the triplex-forming molecule is a tail clamp peptide nucleic acid with the sequence N-terminus—Lys-Lys-Lys-HT TJT-OOO-TCT TCC GAG CAG-Lys-Lys-Lys—C terminus (SEQ ID NO: 1) terminus, wherein J=pseudoisocytosine and O=the flexible linker 8-amino-3,6-dioxaoctanoic acid monomers.

12. The recombinagenic or mutagenic composition of claim 1 wherein the donor oligonucleotide has the sequence
(SEQ ID NO: 16)
5′GGGACGGCGCCCACATAGGCCAAATTCAATTGCT
GATCCCAGCTTAAGACGTACTGGTCAGCCTGGC3′

13. The recombinagenic or mutagenic composition of claim 1 wherein the target sequence of the triplex-forming molecule contains part or all of the sequence 5′ CCTTCACCAAGGGGA 3′ (SEQ ID NO:6).

14. The recombinagenic or mutagenic composition of claim 1 wherein the triplex-forming molecule is a tail clamp peptide nucleic acid with the sequence N-terminus—Lys-Lys-Lys-T TJJ JJT-OOO-TCC CCT TGG TGA AGG -Lys-Lys-Lys—C terminus (SEQ ID NO: 5), wherein J=pseudoisocytosine and O=the flexible linker 8-amino-3,6-dioxaoctanoic acid monomers.

15. The recombinagenic or mutagenic composition of claim 1 wherein the donor oligonucleotide has the sequence 5′ AGGACGGTCCCGGCCTGCGACACTTCCGCCCATAATTGTTCTTCATCT GCGGGGCGGGGGGGGG 3′ (SEQ ID NO: 15).

16. A method of treating of a lysosomal storage disorder in subjects with one or more mutations in one or more human genes encoding an enzyme necessary for the metabolism of lipids, glycoproteins, or mucopolysaccharides comprising administering the composition of claim 1 to an individual in need of treatment thereof.

17. The method of claim 16 comprising
a) isolating cells from a host,
b) contacting the cells ex vivo with the composition of claim 1 wherein the donor oligonucleotide comprises one or more nucleotide mutations, deletions or insertions relative to the target duplex DNA nucleotide sequence,
c) expanding the cells in culture, and
d) administering the cells to a subject in need thereof.

18. The method of claim 17 wherein the cells are synchronized in S-phase to further increase the frequency of gene correction.

19. The method of claim 16 wherein the defect is selected from the group consisting of defects causing Gaucher's disease, Fabry disease, and Hurler syndrome.

20. A method of determining the frequency of correction of a gene encoding an enzyme comprising
a) contacting a population of cells ex vivo with the composition of claim 1 wherein the donor oligonucleotide comprises one or more nucleotide mutations, deletions or insertions relative to the target duplex DNA nucleotide sequence,
b) expanding the cells in culture,
c) isolating protein from the cells,
d) applying protein to an enzyme assay, and
e) comparing the results to a standard curve

21. A method of determining the identifying cells with corrected enzymatic function comprising
a) contacting a population of cells ex vivo with the composition of claim 1 wherein the donor oligonucleotide comprises one or more nucleotide mutations, deletions or insertions relative to the target duplex DNA nucleotide sequence,
b) isolating individual clones from the population
b) expanding the clones in culture,
c) isolating protein from each clonal population,
d) applying protein to an enzyme assay, and
e) comparing the results to a standard curve

22. A method of claim 20 wherein the donor oligonucleotide target sequence contain one or more mutations in the α-L-iduronidase gene and the enzyme assay is a 4-methylumbelliferyl α-Iduronide (4MU) assay.


SEQUENCE LISTING (30 sequences)

SEQ ID NO: 1 - DNA, 12 bp, Artificial Sequence
  synthetic peptie nucleic acid
tcttccgagcag

SEQ ID NO: 2 - DNA, 12 bp, Homo sapiens
ctgctcggaaga

SEQ ID NO: 3 - DNA, 24 bp, Homo sapiens
tgggggctgctcggaagacccctt

SEQ ID NO: 4 - DNA, 24 bp, Homo sapiens
aaggggtcttccgagcagccccca

SEQ ID NO: 5 - DNA, 15 bp, Artificial Sequence
  synthetic peptide nucleic acid
tccccttggtgaagg

SEQ ID NO: 6 - DNA, 15 bp, Homo sapiens
ccttcaccaagggga

SEQ ID NO: 7 - DNA, 27 bp, Homo sapiens
gggactccttcaccaaggggaggggga

SEQ ID NO: 8 - DNA, 27 bp, Homo sapiens
tccccctccccttggtgaaggagtccc

SEQ ID NO: 9 - DNA, 27 bp, Homo sapiens
ctcagctgggactagcagctcaacctc

SEQ ID NO: 10 - DNA, 27 bp, Artificial Sequence
  sequence introduced by wildtype codon modifier (WT CM)
ttaagctgggatcagcaattgaatttg

SEQ ID NO: 11 - DNA, 21 bp, Homo sapiens
gaggagcagctctaggccgaa

SEQ ID NO: 12 - DNA, 21 bp, Artificial Sequence
  sequence introduced by a wildtype codon modifier (WT CM)
gaagaacaattatgggcggaa

SEQ ID NO: 13 - DNA, 12 bp, Homo sapiens
tcttccgagcag

SEQ ID NO: 14 - DNA, 15 bp, Homo sapiens
tccccttggtgaagg

SEQ ID NO: 15 - DNA, 64 bp, Artificial Sequence
  donor oligonucleotide targeting the W402X mutation
aggacggtcccggcctgcgacacttccgcccataattgttcttcatctgcggggcggggggggg

SEQ ID NO: 16 - DNA, 67 bp, Artificial Sequence
  donor oligonucleotide targeting the Q70X mutation
gggacggcgcccacataggccaaattcaattgctgatcccagcttaagacgtactggtcagcctggc

SEQ ID NO: 17 - DNA, 46 bp, Artificial Sequence
  synthetic PCR forward primer “70PNASDMF”
gacagcaagggggaggattgctgctcggaagacaatagcaggcatg

SEQ ID NO: 18 - DNA, 46 bp, Artificial Sequence
  synthetic PCR reverse primer “70PNASDMR”
catgcctgctattgtcttccgagcagcaatcctcccccttgctgtc

SEQ ID NO: 19 - DNA, 31 bp, Artificial Sequence
  synthetic PCR primer
cggtgcggatccgctgcggggagcgcacttc

SEQ ID NO: 20 - DNA, 18 bp, Artificial Sequence
  synthetic PCR primer
gtgtcgtcgctcgcgtag

SEQ ID NO: 21 - DNA, 28 bp, Artificial Sequence
  synthetic IDUA primer for allele specific PCR
tggcggggcctggggactccttcaccaa

SEQ ID NO: 22 - DNA, 24 bp, Artificial Sequence
  synthetic IDUA primer for allele specific PCR
gcgggtgtcgtcgctcgcgtagat

SEQ ID NO: 23 - DNA, 23 bp, Artificial Sequence
  synthetic IDUA primer for allele specific PCR
gaagaacaattatgggcggaagt

SEQ ID NO: 24 - DNA, 20 bp, Artificial Sequence
  synthetic IDUA primer for allele specific PCR
cctgggggcggtgggcgctg

SEQ ID NO: 25 - DNA, 24 bp, Artificial Sequence
  synthetic IDUA primer for allele specific PCR
cgctgccagccatgctgaggctcg

SEQ ID NO: 26 - DNA, 24 bp, Artificial Sequence
  synthetic IDUA primer for allele specific PCR
acacagggatgctcacgggtgcac

SEQ ID NO: 27 - DNA, 27 bp, Artificial Sequence
  synthetic IDUA primer for allele specific PCR
ttaagctgggatcagcaattgaatttg

SEQ ID NO: 28 - DNA, 21 bp, Artificial Sequence
  synthetic IDUA primer for allele specific PCR
acagccagcaaggacacgctc

SEQ ID NO: 29 - DNA, 12 bp, Artificial Sequence
  synthetic peptide nucleic acid
tcttccgagcag

SEQ ID NO: 30 - DNA, 15 bp, Artificial Sequence
  synthetic peptide nucleic acid
tccccttggtgaagg